SEQUENCE LISTING (10 sequences)

SEQ ID NO: 1 - protein, 34 aa, synthetic construct
SVSEIQLMHNLGKHLNSMERVEWLRKKLQDVHNF

SEQ ID NO: 2 - protein, 36 aa, synthetic construct
AVSEHQLLHDKGKSIQDLRRRFFLHHLIAEIHTAEI

SEQ ID NO: 3 - protein, 36 aa, synthetic construct
AVAEIQLMHQRGKSIQDLRRRFFLHHLIAEIHTAEI

SEQ ID NO: 4 - protein, 36 aa, synthetic construct
AVAEIQLMHQRAKWIQDLRRRFFLHHLIAEIHTAEI

SEQ ID NO: 5 - protein, 36 aa, synthetic construct
AVAEIQLMHQRAKWLNSLRRRFFLHHLIAEIHTAEI

SEQ ID NO: 6 - protein, 36 aa, synthetic construct
AVAEIQLMHQRAKWLNSMRRRFFLHHLIAEIHTAEI

SEQ ID NO: 7 - protein, 36 aa, synthetic construct
AVAEIQLMHQRAKWLNSMERVEFLHHLIAEIHTAEI

SEQ ID NO: 8 - protein, 36 aa, synthetic construct
AVAEIQLMHQRAKWLNSMERVEWLRKLIAEIHTAEI

SEQ ID NO: 9 - protein, 36 aa, synthetic construct
AVAEIQLMHQRAKWLNSMERVEWLRKKLQDIHTAEI

SEQ ID NO: 10 - protein, 36 aa, synthetic construct
AVAEIQLMHQRAKWIQDARRRAFLHKLIAEIHTAEI

(19) United States
(12) Patent Application Publication
(10) Pub. No.: US 2025/0032590 A1
(43) Pub. Date: Jan. 30, 2025

(54) PARATHYROID HORMONE COMPOUNDS IN THE TREATMENT OF HYPOPARATHYROIDISM

(71) Applicant: AMOLYT PHARMA, Ecully (FR)

(72) Inventors: Michael Culler, Cambridge, MA (US); Soraya Allas, Ecully (FR); Mark Sumeray, Ecully (FR); Thierry Abribat, Ecully (FR)

(21) Appl. No.: 18/784,809

(22) Filed: Jul. 25, 2024

Related U.S. Application Data
(60) Provisional application No. 63/515,944, filed on Jul. 27, 2023.

(51) Int. Cl. A61K 38/29; A61P 5/18

(52) U.S. Cl. CPC A61K 38/29; A61P 5/18

(57) ABSTRACT

The present technology generally relates to a method for management and/or treatment of hypoparathyroidism (HP) in a subject. The method comprises administering a dose of a PTH compound to the subject, wherein the PTH compound has an amino acid sequence as set forth in SEQ ID NO: 10; and wherein administration of the PTH compound maintains or improves bone integrity.

### CROSS-REFERENCE TO RELATED APPLICATION

This application claims the benefit of and priority to U.S. provisional patent application No. 63/515,944, filed on Jul. 27, 2023; the content of which is herein incorporated in entirety by reference.

### REFERENCE TO SEQUENCE LISTING

This application is being filed with a sequence listing in electronic format. The sequence listing is provided as a file entitled BCF002.003AUSSequenceListing.XML, created on Jul. 25, 2024, which is approximately 10,018 bytes in size. The information in the electronic format of the sequence listing is incorporated herein by reference in its entirety.

### TECHNICAL FIELD

The present technology relates to the use of parathyroid hormone compounds in the treatment of hypoparathyroidism.

Hypoparathyroidism (HP) is a rare disease characterized by absence or inappropriately low concentrations of circulating parathyroid hormone (PTH), leading to hypocalcaemia, hyperphosphatemia, and elevated fractional excretion (FECa) of calcium in the urine or hypercalciuria.

The physiological role of PTH is to maintain the homeostatic balance of serum calcium, which plays a critical role in a number of biological processes. In this tightly regulated process, low serum calcium is registered by calcium-sensing receptors on parathyroid cells, which, in turn, stimulate the parathyroid glands to release PTH into circulation. Signaling is modulated by the PTH receptor at multiple distal sites, resulting in increased absorption of dietary calcium in the gut (i.e., by converting 25-hydroxy vitamin D to 1,25-dihydroxy vitamin D), increased reabsorption of calcium into the blood prior to excretion by the kidneys, and release of calcium from the mineral-rich deposits of the bone matrix (Ref. 1).

Clinical presentation of HP reflects impact on a large number of tissues and organ systems, including the muscles, brain, heart, and kidneys and varies from mild disease, with paresthesia (burning or tingling sensation) and muscle cramps, to severe symptoms, such as laryngospasm and seizures (Ref. 2). In HP, bone turnover is decreased with reduced biochemical markers of bone resorption and formation. The reduced bone turnover in HP results in increased average bone mineral density as compared to age and sex-matched controls, but profoundly abnormal bone architecture due to the accumulation of damaged or overly mature bone (Ref. 3). While the increased bone density and decreased bone quality appears to create a balance such that risk of bone fracture in HP patients appears similar to that of normal subjects in population studies (Refs. 13, 14), it is logical to assume that any treatment that decreases bone density in HP patients could increase the risk of fracture from poor quality bone architecture. Recent studies have, in fact, demonstrated increased morphometric vertebral fractures in hypoparathyroid patients (Refs. 15, 16) as compared to matched normal subjects.

About 80% of the approximately 80,000 people in the U.S. and 110,000 in the European Union with HP are women. Despite available treatments, patients experience persistent, life-altering symptoms and often develop complications and comorbidities that diminish quality of life (QoL) and create segments of the patient population with specific clinical needs. Approximately 17% of patients with hypoparathyroidism have osteopenia or osteoporosis and 53% are peri- or postmenopausal women with an increased risk of developing osteoporosis. For these patients, any bone loss is deleterious. In addition, approximately half of these patients have hypercalciuria, that is responsible for the development of nephrocalcinosis, kidney stones, and progressive kidney disease. As a results, approximately 26% of patients with hypoparathyroidism have chronic kidney disease or failure, highlighting the importance of reducing urinary calcium excretion as a key treatment goal.

The primary goals in treating and managing patients with HP are: i) maintaining stable serum calcium concentration at normal levels for 24 hours, thereby improving or preventing signs and symptoms of HP for 24 hours; ii) normalizing urinary calcium excretion in patients with hypercalciuria, or elevated levels of calcium in the urine, to avoid related impairment of renal function, nephrocalcinosis, and chronic kidney disease; and iii) preserving bone integrity and bone mass in order to prevent increasing patients' risk of fracture.

Conventional therapies for patients with HP include calcium supplements and activated vitamin D. These supplements provide only short-term control of serum calcium levels and do not adequately control symptoms. As a result, HP patients often need to take substantial amounts of supplements throughout the day (as many as 10 to 15 or more tablets a day) to adequately control serum calcium levels. In addition, calcium/vitamin D supplementation does not improve the renal reabsorption of calcium which is deficient in HP patients, and, used chronically, exacerbates potential deleterious effects on the kidney. The continuous flushing of calcium through the kidney, made greater by calcium supplementation, is toxic to the tissues and greatly increases the chance of chronic kidney disease, kidney stones, and impaired kidney function. This is compounded by the increased risk of kidney stones that can lead to kidney injury due to infection and obstruction of urinary outflow.

Natpara™, which is recombinant natural human PTH ((rh)PTH(1-84)) has been approved by the FDA and EMA as an adjunct treatment to calcium and vitamin D supplements to control hypocalcemia in patients with HP. The drug however has a short half-life and does not control calcium levels for the entire day. Clinical studies have failed to demonstrate reduction of urinary calcium excretion or improvement in quality of life (Refs. 5, 6). While short exposure with intermittent administration of PTH was shown to increase bone volume, clinical experience with (rh)PTH(1-84) has shown a lack of 24-hour control of serum calcium and elevated urinary calcium excretion in many subjects and the presence of adverse effects such as hyper/hypocalcemia and vasoactive events (Ref. 5).

In order to overcome the limitations of Natpara™, sustained, long-exposure PTH formulations have been developed. One of them, TransCon™PTH, a sustained-release, injectable prodrug formulation of native PTH (1-34), provides stable serum calcium levels that may improve the control of some disease symptoms and, in addition, may enhance renal reabsorption of calcium in patients, thereby reducing the risk of kidney disease. TransCon™PTH, is currently in late-stage clinical development by Ascendis Pharma A/S.

However, continuous, non-pulsatile administration of PTH has been observed in animal models and in clinical trials to spark safety issues, most notably bone resorption, which raises concern for patients with HP, who are mostly peri- and menopausal women with already heightened risk of osteoporosis (Refs. 17, 18, 19).

Treatment of TPTX rats with TransCon™PTH was shown to produce a continuous, non-pulsatile, infusion-like pharmacokinetic profile, and a marked decrease in bone mineral density (BMD) was observed compared to both the sham-operated and TPTX rats treated with the vehicle alone (Ref. 10). When assessing bone mineral density, a decrease in mean T-scores at 52-week treatment in human HP patients with TransCon™PTH was observed (Ref. 12 and Ascendis web page, https://ascendispharma.gcs-web.com/, July 2023, incorporated herein by reference).

TransCon™PTH has been shown to decrease mean 24-hour urine calcium levels in patients with HP. However, data from Phase 3 trial indicate show that a number of patients are still hypercalciuric after 6 months of treatment (Ref. 11, FIG. 7).

In view of the above, there remain unmet needs for treatments of hypoparathyroidism capable of maintaining steady, normal blood calcium levels, restoring normal reabsorption of calcium by the kidney in hypercalciuric patients and preserving bone integrity and bone mass. None of the treatments for HP currently available or in clinical development have been shown to provide a comprehensive therapeutic effect across all these treatment goals.

### SUMMARY

It is an object of the present technology to ameliorate at least some of the deficiencies present in the prior art.

According to one aspect, the present technology relates to a method for management and/or treatment of hypoparathyroidism (HP) in a subject. The method comprises administering a dose of a PTH compound to the subject, wherein the PTH compound has an amino acid sequence as set forth in SEQ ID NO: 10; and wherein administration of the PTH compound maintains or improves bone integrity.

According to one aspect, the present technology relates to a method for normalizing urine calcium level in a hypercalciuric subject afflicted with hypoparathyroidism. The method comprises administering a dose of a PTH compound to the subject, wherein the PTH compound has an amino acid sequence as set forth in SEQ ID NO: 10.

According to one aspect, the present technology relates to a method for maintaining steady state blood calcium levels in subject afflicted with hypoparathyroidism. The method comprises administering a dose of a PTH compound to the subject, wherein the PTH compound has an amino acid sequence as set forth in SEQ ID NO: 10.

According to one aspect, the present technology relates to a method for managing or treating hypoparathyroidism in a subject. The method comprises: i) administering a dose of a PTH compound to the subject, wherein the PTH compound has an amino acid sequence as set forth in SEQ ID NO: 10; and ii) titrating the subject off of standard of care treatment for hypoparathyroidism.

According to one aspect, the present technology relates to a method for restoring bone turnover in a subject with hypoparathyroidism. The method comprises administering a dose of PTH compound to the subject, wherein the PTH compound has an amino acid sequence as set forth in SEQ ID NO: 10.

According to one aspect, the present technology relates to a method for managing or treating hypoparathyroidism in a subject, wherein the method i) maintains serum calcium levels within normal range without the need of oral calcium and activated vitamin D supplementation; ii) normalizes 24 hr-urinary calcium; and iii) maintains bone integrity, the method comprising administering a dose of PTH compound to the subject, wherein the PTH compound has an amino acid sequence as set forth in SEQ ID NO: 10.

According to one aspect, the present technology relates to a method for managing or treating hypoparathyroidism in a population of subjects, wherein the method i) maintains serum calcium levels within normal range without the need of oral calcium and activated vitamin D supplementation; ii) normalizes 24 hr-urinary calcium; and iii) maintains bone integrity, the method comprising administering a dose of PTH compound to subjects in the population of subjects, wherein the PTH compound has an amino acid sequence as set forth in SEQ ID NO: 10.

According to one aspect, the present technology relates to a pharmaceutical composition comprising a dose of a PTH compound having an amino acid sequence as set forth in SEQ ID NO: 10, wherein the dose is between 10 μg/day and 120 μg/day. In some instances, the pharmaceutical composition comprises a dose of PTH compound of between 20 μg/day and 120 μg/day.

Additional and/or alternative features, aspects, and advantages of implementations of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a graph showing serum calcium levels over time in parathyroidectomized (PTX) mouse model following administration of the indicated doses of AZP-3601 and of PTH (1-34).

FIG. 2 is a graph showing serum calcium levels in thyroid-paratthyroidectomized (TPTX) rat model administered with the indicated doses of AZP-3601 for a period of 28 days.

FIG. 3 is a graph showing urinary calcium levels in PXT mouse model following administration of the indicated doses of AZP-3601.

FIG. 4 is a graph showing urinary calcium levels in TPXT rat model treated with repeated doses of AZP-3601.

FIG. 5 are graphs showing serum calcium levels in non-human primate up to 96 hours following administration of the indicated doses of AZP-3601, PTH (1-84) and PTH (1-34).

FIGS. 6A, 6B and 6C are graphs showing a direct comparison of distal femur in TPTX rats following 14-day treatment with either daily PTH (1-34) injection (FIG. 6A), continuous PTH (1-34) infusion (FIG. 6B), or daily AZP-3601 injection (FIG. 6C), at doses that normalize serum calcium.

FIG. 7 is a graph showing the effects on bone mineral density in TPTX rat of daily administration of AZP-3601.

FIGS. 8A, 8B, 8C and 8D are graphs showing that daily treatment of healthy non-human primates with AZP-3601 has no significant effect on either anabolic or catabolic bone biomarkers. FIG. 8A (males) and 8B (females) show evolution of the catabolic bone biomarker, CTX, over 39 weeks of daily AZP-3601 administration. FIG. 8C (males) and 8D (females) show evolution of the anabolic bone biomarker, P1NP, over 39 weeks of daily AZP-3601 administration.

FIGS. 9A, 9B, 9C, 9D, 9E, 9F are graphs showing bone mineral density (BMD) by quantitative computed tomography (qCT) after a 39-weeks treatment with the indicated doses of AZP-3601 in non-human primates. FIG. 9A shows femur BMD in males;

FIG. 9B shows femur BMD in females; FIG. 9C shows tibia BMD in males; FIG. 9D shows tibia BMD in females; FIG. 9E shows L4 BMD in males; FIG. 9F shows L4 BMD in females.

FIG. 10 is a graph showing that administration of AZP-3601 to normal, healthy subjects over 14 days causes a dose dependent and sustained steady state increase in serum calcium levels (Multiple ascending dose cohort: 5 cohorts/n=8-10 per cohort).

FIGS. 11A and 11B are graphs showing the effects of administration of AZP-3601 allowed to eliminate the need for calcitriol supplementation. FIG. 11A shows reduction of calcitriol supplementation over 84 days of administration of AZP-3601 (with a starting dose of 20 mg/day) in C1 hypoparathyroid patients who completed the extension period, N=10. FIG. 11B shows reduction of calcitriol supplementation over 84 days of administration of AZP-3601 (with a starting dose of 10 mg/day) in C2 hypoparathyroid patients who completed the extension period, N=14.

FIGS. 12A and 12B are graphs showing the potential of administration of AZP-3601 on elimination of oral calcium intake as part of a standard of care treatment. FIG. 12A shows reduction of oral calcium intake (mg/day) over 84 days of administration of AZP-3601 (with a starting dose of 20 mg/day) in C1 hypoparathyroid patients who completed the extension period, N=10. FIG. 12B shows reduction of oral calcium intake (mg/day) over 84 days of administration of AZP-3601 (with a starting dose of 10 mg/day) in C2 hypoparathyroid patients who completed the extension period, N=14.

FIGS. 13A and 13B are graphs showing that administration of AZP-3601 maintains mean serum calcium within target range. FIG. 13A shows maintained mean serum calcium levels over 84 days of administration of AZP-3601 (with a starting dose of 20 mg/day) in C1 hypoparathyroid patients who completed the extension period, N=10. FIG. 13B shows maintained mean serum calcium levels over 84 days of administration of AZP-3601 (with a starting dose of 10 mg/day) in C2 hypoparathyroid patients who completed the extension period, N=14.

FIGS. 14A and 14B are graphs showing that administration of AZP-3601 induces a rapid, profound, and sustained reduction and normalization of mean 24-hour urine calcium. FIG. 14A shows evolution of 24 h uCa (mg/24 h) over 84 days of AZP-3601 administration in C1 hypoparathyroid patients who completed the extension period, N=10.

FIG. 14B shows evolution of 24 h uCa (mg/24 h) over 84 days of AZP-3601 administration in C2 hypoparathyroid patients who completed the extension period, N=14.

FIGS. 15A and 15B are graphs showing that in hypoparathyroid subjects with hypercalciuria at baseline, administration of AZP-3601 induces a rapid, profound reduction and sustained normalization of mean 24-hour urine calcium. FIG. 15A shows evolution of 24 h uCa (mg/24 h) over 84 days of AZP-3601 administration in C1 hypoparathyroid patients who completed the extension period, N=6. FIG. 15B shows evolution of 24 h uCa (mg/24 h) over 84 days of AZP-3601 administration in C2 hypoparathyroid patients who completed the extension period, N=7.

FIGS. 16A-16D are graphs showing that treatment with AZP-3601 induces a gradual increase on both anabolic and catabolic bone biomarkers to the mid-normal level by 4-8 weeks. FIG. 16A shows evolution of the catabolic bone biomarker, CTx, over 84 days of AZP-3601 administration in C1 hypoparathyroid patients who completed the extension period, N=10. FIG. 16B shows evolution of the anabolic bone biomarker, P1NP, over 84 days of AZP-3601 administration in C1 hypoparathyroid patients who completed the extension period, N=10. FIG. 16C shows evolution of the catabolic bone biomarker, CTx, CTx over 84 days of AZP-3601 administration in C2 hypoparathyroid patients who completed the extension period, N=14. FIG. 16D shows evolution of the anabolic bone biomarker, P1NP, over 84 days of AZP-3601 administration in C2 hypoparathyroid patients who completed the extension period, N=14.

FIGS. 17A and 17B are graphs showing that bone mineral density and trabecular bone score remains stable upon AZP-3601 administration. FIG. 17A shows that bone mineral density (BMD) remains stable in AZP-3601-administered hypoparathyroid subjects over 84 days of treatment. FIG. 17B shows that trabecular bone score (TBS) remains stable in AZP-3601-administered hypoparathyroid subjects over 84 days of treatment.

FIGS. 18A and 18B are graphs showing the effect of AZP-3601 administration on Z-score and T-score bone. FIG. 18A shows Z-score, which compares the BMD measured in patients to age- and sex-match healthy subjects. FIG. 18B shows T-score, which compares the BMD in patients to the BMD from young healthy subjects.

### DESCRIPTION OF EMBODIMENTS

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

The use of “including”, “comprising”, or “having”, “containing”, “involving” and variations thereof herein, is meant to encompass the items listed thereafter as well as, optionally, additional items. In the following description, the same numerical references refer to similar elements.

As used herein the expression “standard of care” or “SOC” refers to oral administration of calcium and active vitamin D.

As used herein the expression “titrating off of standard of care” refers to decreasing and/or removing oral calcium and active vitamin D administration.

As used herein, the expression “normal calcium levels” refers to a serum calcium level of between 8.3 mg/dL to 10.6 mg/dL or between 2.075 mmol/L to 2.65 mmol/L. In humans, the normal level in certain instances corresponds to a serum calcium level of above 8.5 mg/dL (albumin-adjusted).

The expression “albumin-adjusted calcium levels” means that the measured serum calcium level is corrected for calcium bound to albumin according to the following formula: albumin-adjusted serum calcium (mg/dL)=measured total Ca (mg/dL)+0.8*(4.0-serum albumin [g/dL]).

As used herein, the expression “normal urine calcium levels” refers to urine calcium levels between 100 to 300 milligrams per day (mg/day) or 2.50 to 7.50 millimoles per 24 hours (mmol/24 hours). For a diet low in calcium, the amount of calcium in the urine will be 50 to 150 mg/day or 1.25 to 3.75 mmol/24 hours.

As used herein, the term “hypercalciuria” refers to an excess of calcium in the urine. It may be secondary, that is, a side-effect of some other condition causing high levels of calcium in the bloodstream (e.g., HP) or it may be “idiopathic” occurring on its own, with normal blood calcium levels. Typically, hypercalciuria refers to urine calcium levels of more than 250 mg/24 hr in women; over 300 mg/24 hr in men.

As used herein, the expression “bone mineral density (BMD)” refers to the amount of bone mineral in bone tissue. The concept is of mass of mineral per volume of bone (relating to density in the physics sense). Bone density measurement is used in clinical settings as an indirect indicator of osteoporosis and fracture risk. It is measured by a procedure called densitometry. The T-score is the relevant measure when screening for osteoporosis. It is the bone mineral density at the site when compared to the “young normal reference mean”. It is a comparison of a subject's bone mineral density to that of a healthy 30-year-old. Normal is a T-score of −1.0 or higher. Osteopenia is defined as between −1.0 and −2.5. Osteoporosis is defined as −2.5 or lower, meaning a bone density that is two and a half standard deviations below the mean of a 30-year-old man/woman. The Z-score for bone density is the comparison to the “age-matched normal” and is usually used in cases of severe osteoporosis. There is a statistical association between poor bone density and higher probability of fracture. Fractures of the legs and pelvis due to falls are a significant public health problem, especially in elderly women, leading to much medical cost, inability to live independently and even risk of death. Bone density measurements are used to screen people for osteoporosis risk and to identify those who might benefit from measures to improve bone strength.

The expression “parathyroid hormone compound” or “PTH compound”, as used herein, refers to PTH polypeptides, as well as variants, analogs, orthologs, homologs, derivatives, and fragments thereof. The expression “PTH compound” also refers to PTH-related polypeptides (PTHrP), such as the polypeptide identified in Table 1 below, that bind to and activate the common PTH/PTHrPl receptor. Other PTH compounds have been discussed in U.S. Pat. No. 9,492,508, the content of which is incorporated herein by reference.

TABLE 1
AMINO ACID SEQUENCES OF PTH COMPOUNDS
OF THE PRESENT TECHNOLOGY.

| SEQ ID | | |
|---|---|---|
| NO: | Compound Name | Amino acid sequence |
| 1 | PTH(1-34) | ¹SVSEIQLMHNLGKHLNSMERVEWLRKKLQDVHNF³⁴ |
| 2 | PTHrP(1-36) | ¹AVSEHQLLHDKGKSIQDLRRRFFLHHLIAEIHTAEI³⁶ |
| 3 | M-PTH(1-11)/PTHrp(12-36) | ¹AVAEIQLMHQRGKSIQDLRRRFFLHHLIAEIHTAEI³⁶ |
| 4 | M-PTH(1-14)/PTHrp(15-36) | ¹AVAEIQLMHQRAKWIQDLRRRFFLHHLIAEIHTAEI³⁶ |
| 5 | M-PTH(1-17)/PTHrp(18-36) | ¹AVAEIQLMHQRAKWLNSLRRRFFLHHLIAEIHTAEI³⁶ |
| 6 | M-PTH(1-18)/PTHrp(19-36) | ¹AVAEIQLMHQRAKWLNSMRRRFFLHHLIAEIHTAEI³⁶ |
| 7 | M-PTH(1-22)/PTHrp(23-36) | ¹AVAEIQLMHQRAKWLNSMERVEFLHHLIAEIHTAEI³⁶ |
| 8 | M-PTH(1-26)/PTHrp(27-36) | ¹AVAEIQLMHQRAKWLNSMERVEWLRKLIAEIHTAEI³⁶ |
| 9 | M-PTH(1-30)/PTHrp(31-36) | ¹AVAEIQLMHQRAKWLNSMERVEWLRKKLQDIHTAEI³⁶ |
| 10 | M-PTH(1-14)/PTHrP(15-36) | ¹AVAEIQLMHQRAKWIQDARRRAFLHKLIAEIHTAEI³⁶ |

The expression “PTH compound” as sued herein also includes poly(amino acid) conjugates which have a sequence as described above, but having a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in proteins and peptides, or to any carbon atom-comprising moiety attached to the backbone of a poly(amino acid) conjugate, such as for example in the case of depsipeptides.

The term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

In one embodiment, the PTH compound of the present technology is a synthetic 36-amino acid hybrid peptide analogue of human PTH and PTHrP that has been designed to potently bind to the R⁰ conformation of the PTH1 receptor, while having a short circulating half-life (Ref. 5). The high affinity of the PTH compound of the present technology for the distinct PTH1 receptor conformation R⁰ maintains binding of the ligand through multiple rounds of G-protein coupling and activation resulting in a greatly prolonged signal transduction and cellular response (i.e., prolonged duration of action on calcium metabolism). The brief circulating half-life is intended to decrease the potential of prolonged PTH receptor exposure that would promote adverse effects on bone (i.e., bone resorption) and contribute to cardiovascular safety events such as orthostatic hypotension.

In certain embodiments, the PTH compound of the present technology is a peptide having the amino acid sequence as set forth in SEQ ID NO: 1, 2, 3, 4, 5, 6, 7, 8, 9 or 10.

In certain embodiments, the PTH compound of the present technology is a peptide having an amino acid sequence as set forth in SEQ ID NO: 10, also referred to herein as “AZP-3601”.

Pharmacology effects of AZP-3601 have been observed in rodent models of HP and in the normal monkey. In hypoparathyroid mice, a single subcutaneous (sc) injection of AZP-3601 at doses ranging from 10 nmol/kg to 20 nmol/kg increased serum calcium to normal levels for up to 72 hours, and decreased serum inorganic phosphorus to normal levels for over 8 hours, whereas PTH(1-34), injected at a higher dose increased serum calcium and decreased serum inorganic phosphorus much more transiently (<8 hours for serum calcium and <4 hours for serum inorganic phosphorus) (Ref. 7). Similar results were also observed in hypoparathyroid/hypothyroid rats in which it was demonstrated that chronic treatment with AZP-3601 for 28 days restored serum calcium and serum inorganic phosphorus without increasing urinary calcium excretion (Ref. 8).

AZP-3601 administration in non-human primates shows that a significant and maximal effect was measured 24 hours after AZP-3601 injection at 2.1 μg/kg to 4.3 μg/kg. Highest dose levels of 10.7 μg/kg or 42.7 μg/kg induced significant hypercalcemia, and serum calcium remained elevated for at least 4 days post injection (Ref. 8).

The present technology stems from the investigators' surprising findings that daily administration of a PTH compound, in particular a long-acting PTH compound such as AZP-3601 restores serum calcium levels to normal levels (8.3 to 10.6 mg/dL or 2.075 to 2.65 mmol/L) and enables withdrawal of SOC within just 28 days of starting the PTH therapy. Surprisingly, the investigators have found that these results were achieved without compromising bone integrity, that is without a reduction in bone mass. The investigators also surprisingly found that daily administration of AZP-3601 normalizes urinary calcium in hypercalciuric subjects. Another surprising aspect of the findings stems from the investigators' observations that the sustained effects were achieved despite the short circulating half-life of AZP-3601.

Therefore, the present investigators have found that daily administration of AZP-3601 produces a neutral effect on bone in subjects with already balanced bone turnover, as pre-clinical rodent models of HP acutely parathyroidectomized. In addition, a balanced anabolic/catabolic effect of daily AZP-3601 was demonstrated to produce balanced, physiological, anabolic/catabolic effect on bone human subjects with established HP and slowed/arrested bone turnover.

In one embodiment, the present technology thus relates to a method for management and/or treatment of hypoparathyroidism (HP) in a subject. The method comprises administering a dose of a PTH compound as defined herein to the subject.

In some instances, the subjects in need of the methods of the present technology are hypoparathyroid subjects (HP subjects). Hypoparathyroidism can lead to hypocalcemia and hyperphosphatemia. As a result, subjects with hypoparathyroidism may experience a range of severe and potentially life-threatening short-term and long-term complications, including neuromuscular irritability, renal complications, and vascular calcifications. Cognitive impairment is also common. Without the regulation of PTH in HP subjects, there is a low bone turn-over state and bone turn-over markers are in the lower half of normal ranges. Consequently, HP subjects have slightly higher bone mineral densities as compared with healthy individuals. HP subjects have slightly higher average BMD as compared with healthy individuals. However, although the average value for BMD is higher than subjects without HP, there is considerable heterogeneity in the HP population, and many patients have lower than average BMD. Many of these patients are post-menopausal females, and may have been developing osteopenia or osteoporosis for several years prior the onset of HP. Furthermore, despite the increased bone density observed in some patients, the abnormal bone microarchitecture in HP subjects may result in decreased resilience. Increased bone density observed in HP subjects does not provide increased protection against fracture risk, as demonstrated in population studies, and is likely due to the counterbalance effect of impaired bone quality associated with this disease. There is one subset of HP subjects who are particularly prone to hypercalciuria: those with autosomal dominant hypoparathyroidism (5, 6, 8, 9). This is because at baseline, before treatment, the constitutively active calcium receptor in the kidney activates renal calcium excretion.

In some instances, the PTH compound is administered daily to the subject. In some instances, the PTH compound is administered once daily to the subject.

In some embodiments, the subjects in need of administration of a PTH compound in accordance with the method of the present technology include subjects who are at risk or who are experiencing an increase in bone loss. In some embodiments, the subjects in need of administration of a PTH compound in accordance with the method of the present technology include subjects who are at risk or who are experiencing a decrease in bone integrity. In some instances, the subjects in need of administration of a PTH compound in accordance with the present technology are hypercalciuric subjects (i.e., demonstrate excess calcium in the urine). In some embodiments, the subjects in need of administration of a PTH compound in accordance with the method of the present technology include subjects who are at risk or who are experiencing an increase in bone loss and who are hypercalciuric. In some instances, the subjects in need of administration of a PTH compound in accordance with the present technology are peri-menopausal women and post-menopausal women. In some other instances, the subjects suffer from a bone loss-related condition such as osteopenia and osteoporosis.

In one embodiment, the method of the present technology further comprises administering a PTH compound to a subject while titrating the subject off of standard of care for HP.

In some embodiments, titrating the subject off of standard care is performed by decreasing the daily intake of oral calcium and decreasing the daily intake of active vitamin D. In some other implementations, titrating the subject off of standard of care is performed until the administration of the daily dose of the PTH compound results in a stable albumin-corrected serum calcium level in the subject.

In some embodiments, titrating the subject off of standard care is performed in a stepwise approach until the administration of vitamin D is eliminated and until the administration of calcium is reduced to or reduced below 600 mg/day.

In some embodiments, the methods of the present technology comprise titrating the subject off of standard of care within 12 weeks from the time the first dose of the PTH compound is administered. In some embodiments, the methods of the present technology comprise titrating the subject off of standard of care within 10 weeks from the time the first dose of the PTH compound is administered. the methods of the present technology comprise titrating the subject off of standard of care within 8 weeks from the time the first dose of the PTH compound is administered. the methods of the present technology comprise titrating the subject off of standard of care within 6 weeks from the time the first dose of the PTH compound is administered. the methods of the present technology comprise titrating the subject off of standard of care within 4 weeks from the time the first dose of the PTH compound is administered. the methods of the present technology comprise titrating the subject off of standard of care within 2 weeks from the time the first dose of the PTH compound was administered. Various titration schemes are suitable. In certain embodiments, titrating the patients off of standard of care involves a stepwise reduction followed by complete omission of orally administered active vitamin D, followed by a stepwise reduction followed by complete omission of orally administered calcium. It is understood that some subject's diets do not allow a sufficient nutritional uptake of calcium (usually considered to be <750 mg calcium per day), as may be the case in lactose-intolerant subjects, for example. These subjects continue taking oral calcium supplements, in the form of for example a once daily oral administration of calcium, such as in the form of a calcium tablet. This calcium supplement is however not related to the treatment of hypoparathyroidism and is common practice also in healthy subjects.

In some embodiments, the daily dose of the PTH compound to be administered to the subject ranges between 10 μg/day and 120 μg/day, or between 10 μg/day and 100 μg/day, or between 10 μg/day and 90 μg/day, or between 10 μg/day and 80 μg/day daily, or between 10 μg/day and 70 μg/day, or between 10 μg/day and 60 μg/day or between 10 μg/day and 50 μg/day, or between 20 μg/day and 120 μg/day, or between 20 μg/day and 100 μg/day, or between 20 μg/day and 90 μg/day, or between 20 μg/day and 80 μg/day daily, or between 20 μg/day and 70 μg/day, or between 20 μg/day and 60 μg/day or between 20 g/day and 50 μg/day.

In some embodiments, the daily dose of the PTH compound to be administered to the subject is 120 μg/day. In some embodiments, the daily dose of the PTH compound to be administered to the subject is 100 μg/day. In some other embodiments, the daily dose of the PTH compound to be administered to the subject is 90 μg/day. In some other embodiments, the daily dose of the PTH compound to be administered to the subject is 80 μg/day. In some other embodiments, the daily dose of the PTH compound to be administered to the subject is 70 μg/day. In some other embodiments, the daily dose of the PTH compound to be administered to the subject is 60 μg/day. In some other embodiments, the daily dose of the PTH compound to be administered to the subject is 50 μg/day. In some other embodiments, the daily dose of the PTH compound to be administered to the subject is 40 μg/day. In some other embodiments, the daily dose of the PTH compound to be administered to the subject is 30 μg/day. In some other embodiments, the daily dose of the PTH compound to be administered to the subject is 20 μg/day. In some other embodiments, the daily dose of the PTH compound to be administered to the subject is 10 μg/day.

In some embodiments, the method of the present technology is a stepwise method in which an initial daily dose of PTH is administered to the subject on day 1 of the treatment. In the subsequent days of the treatment, the subject is titrated off of standard of care by decreasing the intake of active vitamin D and calcium until the intake of active vitamin D is no longer required and the intake of calcium is reduced to or below 500 mg/day.

In some instances, the decrease of oral intake of active vitamin D is as follows: a first 50% reduction in oral active vitamin D from baseline dose; a second 75% reduction in oral vitamin D from baseline dose; and a 100% reduction in oral active vitamin D from baseline dose.

In some instances, the decrease of oral intake of calcium is as follows: a first 50% reduction in oral active vitamin D from baseline dose; a second 75% reduction in oral vitamin D from baseline dose; and leaving only 600 mg/day or less in calcium intake.

In some embodiments, the method also comprises increasing the initial dose daily of PTH to achieve the desired reduction of calcium and active vitamin D intake. In some instances, the initial daily dose of the PTH compound is 5 μg/day, 10 μg/day, 15 μg/day, 20 μg/day, 25 μg/day, 30 μg/day, 35 μg/day, 40 μg/day, 45 μg/day, 50 μg/day, 55 μg/day, 60 μg/day, 65 μg/day, 70 μg/day, 75 μg/day, 80 μg/day, 85 μg/day, 90 μg/day, 95 μg/day, 100 μg/day, 110 μg/day, or 120 μg/day. In some instances, the daily dose of the PTH compound to be administered by be increased by 2 μg/day, 5 μg/day, 10 μg/day, 15 μg/day, 20 μg/day, 25 μg/day, 30 μg/day, or more. The increase in PTH compound may be requires after several days or weeks after the administration of the initial dose of the PTH compound.

In some embodiments, the methods of the present technology comprise initiating the treatment with administration of an initial daily dose of the PTH compound defined herein in a concentration of 10 μg/day as a subcutaneous (sc) injection to the subject and to concomitantly decrease the administration of active vitamin D by at least 20%, by at least 25%, by at least 30%, by at least 40%, by at least 45%, or by at least 50%. The method also comprises monitoring serum calcium (and albumin) concentrations every 3 to 7 days after initiation of the treatment (e.g., after administration of the initial dose of the PTH compound) and after each dose change. The dose of the PTH compound may be titrated, for example, every day, every couple days, every week, every 2 weeks, every 3 week, every 4 weeks with the goal to discontinue active vitamin D and to reduce oral calcium supplements to an amount as low as 500 mg/d while keeping serum calcium within the low-normal range. After the initial titration phase when a stable regimen is achieved, serum calcium and phosphate can be monitored every 3 to 6 months and urinary calcium excretion may be monitored yearly.

In other embodiments, the present technology relates to a method for managing or treating hypoparathyroidism in a subject, wherein a first dose of the PTH compound is administered to the subject in a dosage regimen, in which the first dose of the PTH compound is increased in the course of the treatment and wherein such dosage regimen comprises the steps of: i) titrating the first dose of the PTH compound administered to the subject to result in normal serum calcium levels in the subject and maintaining the subject on such first dose for a first time period; ii) increasing the first dose of the PTH compound being administered to the subject for a second time period directly following the first time period to achieve a second dose; and (iii) optionally increasing the second dose of the PTH compound for a third or further subsequent time period to achieve a third dose. In some instances, the third or subsequent dose differs from the second or from the first dose by 2 μg/day, 5 μg/day, 10 μg/day, 15 μg/day, 20 μg/day, 25 μg/day, 30 μg/day, or more. In some instances, the second dose differs from the first dose by 2 μg/day, 5 μg/day, 10 μg/day, 15 μg/day, 20 μg/day, 25 μg/day, 30 μg/day, or more. In certain embodiments the first time period is at least 2 weeks, at least 1 month, at least 2 months, at least 3 months, at least 4 months, at least 5 months, or at least 6 months. In certain embodiments the second time period is at least 1 month, at least 2 months, at least 3 months, or at least 4 months.

In other embodiments, the present technology relates to a method for managing or treating hypoparathyroidism in a subject, wherein the method allows to: i) maintain serum calcium levels within normal range without the need of oral calcium and activated vitamin D supplementation; ii) normalize 24 hr-urinary calcium; and iii) maintain bone integrity, in the subject. The method comprising administering a dose of PTH compound to the subject, wherein the PTH compound has an amino acid sequence as set forth in SEQ ID NO: 10. In some instances, the method allows to maintain serum calcium levels between 8.3 mg/dL to 10.6 mg/dL or between 2.075 mmol/L to 2.65 mmol/L. in some instances, the method allows to normalize 24 hr-urinary calcium to between about 100 mg/day and about 300 mg/day, or between about 100 mg/day and about 250 m/day, or between about 100 mg/day and about 200 mg/day, or between about 100 mg/day and about 150 mg/day, or between about 100 mg/day and about 125 mg/day. In some instances, the method normalizes 24 hr-urinary calcium in the subject within 12 weeks of administration of the PTH compound. In some instances, the method allows to normalize 24 hr-urinary calcium to between about 100 mg/day and about 300 mg/day within 12 weeks of administration of the PTH compound, or between about 100 mg/day and about 250 m/day within 12 weeks of administration of the PTH compound, or between about 100 mg/day and about 200 mg/day within 12 weeks of administration of the PTH compound, or between about 100 mg/day and about 150 mg/day within 12 weeks of administration of the PTH compound, or between about 100 mg/day and about 125 mg/day within 12 weeks of administration of the PTH compound. In some instances, the subjects are hypercalciuric patients.

In some instances, the method allows to maintain bone biomarkers and bone integrity in normal range, thereby restoring bone turnover in HP subjects. As used herein, the expression “bone turnover” refers to the lifelong process whereby mature bone tissue is removed from the skeleton and new bone tissue is formed. These processes also control the reshaping or replacement of bone following injuries like fractures but also micro-damage.

In some embodiments, the present technology relates to a method for managing or treating hypoparathyroidism in a population of subjects, wherein the method allows to: i) maintain serum calcium levels within normal range without the need of oral calcium and activated vitamin D supplementation; ii) normalize 24 hr-urinary calcium; and iii) maintain bone integrity in the subjects of the population. The method comprising administering a dose of PTH compound to the subject, wherein the PTH compound has an amino acid sequence as set forth in SEQ ID NO: 10. In some instances, the method allows to normalize 24 hr-urinary calcium in at least about 90% of the hypercalciuric subjects of the population. In some instances, the method allows to normalize 24 hr-urinary calcium in at least about 85% of the hypercalciuric subjects of the population. In some instances, the method allows to normalize 24 hr-urinary calcium in at least about 80% of the hypercalciuric subjects of the population. In some instances, the method allows to normalize 24 hr-urinary calcium in at least about 75% of the hypercalciuric subjects of the population. In some instances, the method allows to normalize 24 hr-urinary calcium in at least about 70% of the hypercalciuric subjects of the population. In some instances, the method allows to normalize 24 hr-urinary calcium in at least about 65% of the hypercalciuric subjects of the population. In some instances, the method allows to normalize 24 hr-urinary calcium in at least about 60% of the hypercalciuric subjects of the population. In some instances, the method allows to normalize 24 hr-urinary calcium in at least about 55% of the hypercalciuric subjects of the population. In some instances, the method allows to normalize 24 hr-urinary calcium in at least about 50% of the hypercalciuric subjects of the population.

In certain embodiments, the administration of the PTH compound is oral, intravenous, intramuscular, ophthalmic, topical, dermal, subcutaneous, or rectal.

In certain embodiments, the methods of the present technology comprise administering the PTH compound by injection. In certain instance, the PTH compound is administered by subcutaneous injection. In certain instances, the PTH compound is administered once daily by subcutaneous injection.

In some embodiments, the present technology relates to a pharmaceutical composition for management or treatment of hypoparathyroidism in a subject, wherein the pharmaceutical composition comprises a daily dose of a PTH compound. In some implementations, the PTH compound is a peptide having an amino acid sequence as set forth in SEQ ID NO: 10 (AZP-3601).

As used herein the term “pharmaceutical composition” refers to a composition containing one or more active ingredients, such as for example at least one PTH compound (e.g., AZP-3601), and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition for use of the present technology encompasses any composition made by admixing one or more PTH compound and a pharmaceutically acceptable excipient.

As used herein, the term “excipient” refers to a diluent, adjuvant, or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, phenol, amino acids such as methionine and histidine (e.g., L-methionine and L-histidine), mannitol, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesul fonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, citrate, LLC, SNAC, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration. As used herein the term “liquid composition” refers to a mixture comprising a water-soluble PTH compound and one or more solvents, such as water.

The term “drug” as used herein refers to a substance, such as a PTH compound, used in the treatment of hyperparathyroidism. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which comprises a reversible linkage with the biologically active moiety and wherein the specialized protective group alters or eliminates undesirable properties in the parent molecule. This also includes the enhancement of desirable properties in the drug and the suppression of undesirable properties. The specialized non-toxic protective group is referred to as “carrier.” A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

As used herein, the term “reagent” means a chemical compound which comprises at least one functional group for reaction with the functional group of another chemical compound or drug. It is understood that a drug comprising a functional group (such as a primary or secondary amine or hydroxyl functional group) is also a reagent.

In certain embodiments the pharmaceutical composition of the present technology has a pH ranging from and including pH 3 to pH 8. In certain embodiments the pharmaceutical composition has a pH ranging from and including pH 4 to pH 6. In certain embodiments the pharmaceutical composition has a pH ranging from and including pH 4 to pH 5. In some embodiments, the pharmaceutical composition has a pH of 5.6+/−0.3.

In certain embodiments the pharmaceutical composition is a liquid or suspension composition. It is understood that the pharmaceutical composition is a liquid composition if the PTH compound is water-soluble and a suspension formulation if the PTH compound is water-insoluble. In certain embodiments the pharmaceutical composition is a dry formulation which is reconstituted before administration to a patient.

Such liquid, suspension, dry or reconstituted pharmaceutical composition comprises at least one excipient. Excipients used in parenteral formulations may be categorized as, for example, buffering agents, isotonicity modifiers, preservatives, stabilizers, anti-adsorption agents, oxidation protection agents, viscosifiers/viscosity (measured at 1.09 mPa*s for both concentrations of active (250 and 500 μg/mL). enhancing agents, or other auxiliary agents. However, in some cases, one excipient may have dual or triple functions. In certain embodiments the at least one excipient is selected from the group consisting of (i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCCfi may be also used; (ii) Isotonicity modifiers: to minimize pain that can result from cell damage due to osmotic pressure differences at the injection depot; glycerin and sodium chloride are examples; effective concentrations can be determined by osmometry using an assumed osmolality of 282-330 mOsmol/kg for serum (in some instances, the osmolarity may be between or between; (iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride; (iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used; (v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value; (vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used; (vii) Viscosifiers or viscosity enhancers: in case of a suspension retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g., Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection); (viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and (ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

The administration of PTH compound as defined herein may be by any suitable means that results in a concentration of the compound that treats the subject and disease condition. In some instances, the PTH compound may be contained in any appropriate amount in any suitable carrier substance and is generally present in an amount of 0.025% and 1% by weight of the total weight of the composition/dose. The composition may be in the form of, e.g., tablets, ampules, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York, incorporated herein by reference).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastrointestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

The composition comprising the PTH compound described herein may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants.

In some embodiments, the pharmaceutical composition of the present technology is provided in a prefilled pen.

In some embodiments, the prefilled pen comprises between about 20 μg and about 750 μg, between about 20 μg and about 500 μg, between about 20 μg and about 250 μg, or between about 320 μg and about 100 μg of the PTH compound.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### EXAMPLES

### Example 1: Effects of AZP-3601 on Serum Calcium Levels in HP PTX Mouse Model

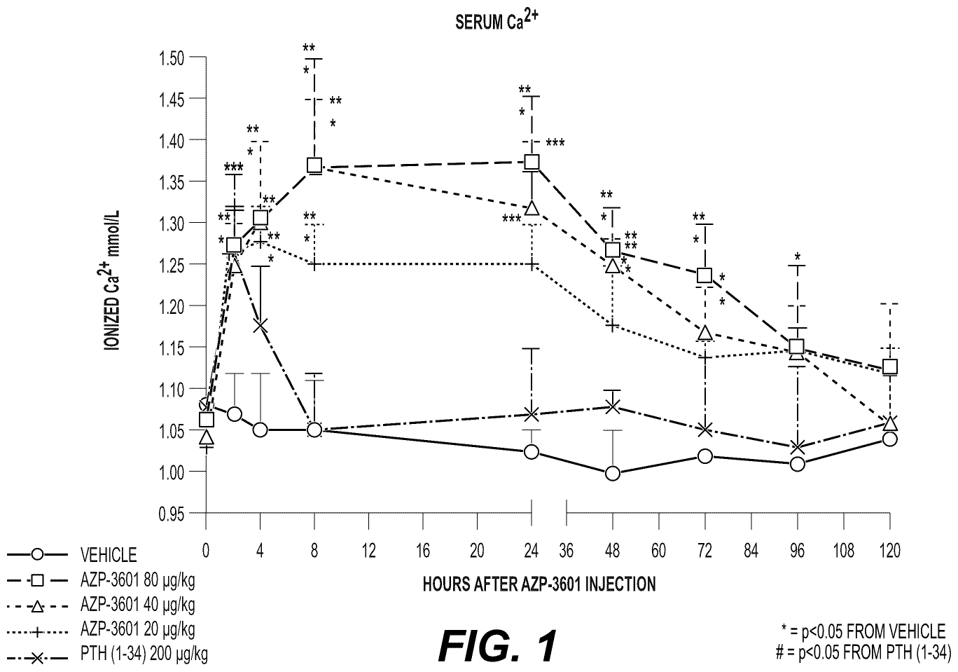

Enhanced effects on serum calcium were observed in preclinical mouse models where HP was induced by parathyroidectomy, or PTX, in which it was observed that AZP-3601 was more potent and effective than natural PTH (1-34) in restoring and maintaining blood calcium and phosphate levels. In FIG. 1, the black line indicates the impact of PTX in mice, resulting in serum calcium levels well below normal levels. Single dosing of PTX mice with PTH (1-34) at 200 μg/kg (blue line) caused a transient elevation in serum calcium, consistent with its short half-life and its weaker interaction with the R⁰ conformation of the PTH1 receptor. By contrast, single dosing of PTX mice with AZP-3601 at 20, 40, or 80 μg/kg (yellow, orange, brown lines, respectively) induced a dose-related normalization of serum calcium that was sustained for up to 72 hours, consistent with its potent interaction with the R⁰ conformation of the PTH1 receptor, and despite AZP-3601 having an equally short half-life as PTH (1-34).

### Example 2: Effects of AZP-3601 on Serum Calcium Levels in HP TPTX Rat Model

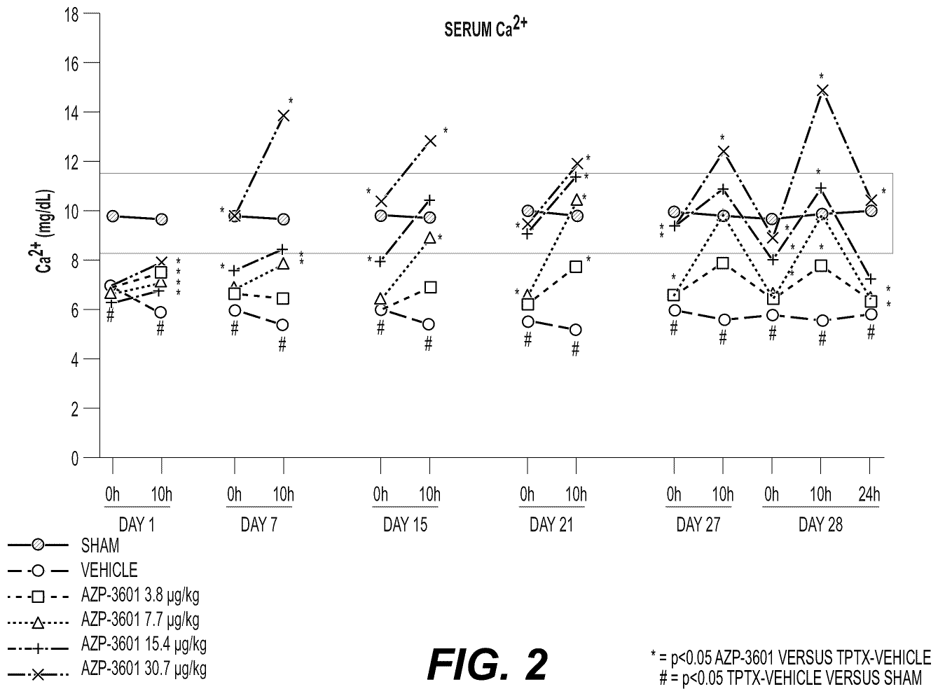

Thyroid-parathyroidectomized, or TPTX, rats were treated with daily subcutaneous injection of AZP-3601 at doses ranging from 3.8 μg/kg/day to 30.7 μg/kg/day. Treatment was continued for 28 days. The chronic treatment with AZP-3601 produced a dose-dependent, progressive normalization of serum calcium levels in the TPTX rat and reduced blood phosphate levels. The increase in calcium accumulated over time (over the first 15 to 21 days), but became more constant thereafter, indicating the development of a steady-state effect (FIG. 2). Due to the cumulative effect, lower doses of AZP-3601 were required to eventually increase blood calcium levels into the normal range as compared to the dose required with a single injection. With repeated administration, a dose of 7.7 μg/kg (1.8 nmol/kg)/day was found to be optimal. This dose normalized blood calcium without increasing urinary calcium excretion, and without producing any significant change in bone. In TPTX rats injected once daily with natural PTH (1-84), there was no persistent increase in blood calcium levels and only marginal reductions in blood phosphate levels, as compared with the levels in vehicle-treated TPTX rats (FIG. 2).

### Example 3: Assessment of AZP-3601 on Urinary Calcium Levels in HP PTX Mice Model

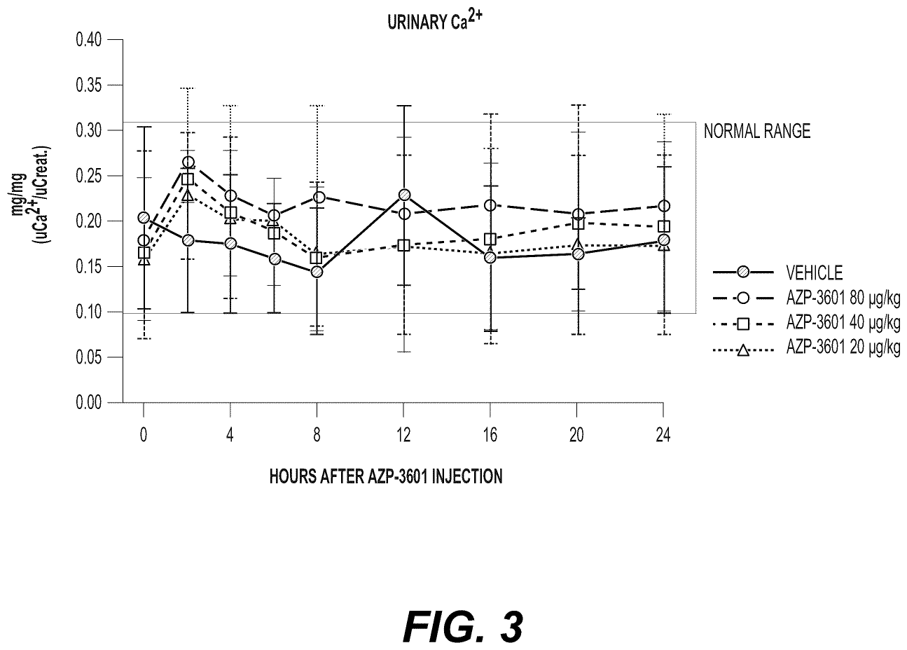

In a preclinical study of PTX mice, single dosing with AZP-3601 resulted in maintenance of normal levels of urinary calcium excretion, despite marked elevation of serum calcium levels (FIG. 3). This data contrasts with the heightened levels of urinary calcium that would be expected from treatment with other approaches to increase serum calcium levels, such as dietary supplementation with inorganic calcium and vitamin D or with continuous infusion of natural PTH.

### Example 4: Assessment of Repeated Doses of AZP-3601 on Urinary Calcium Levels in HP TPTX Rat Model

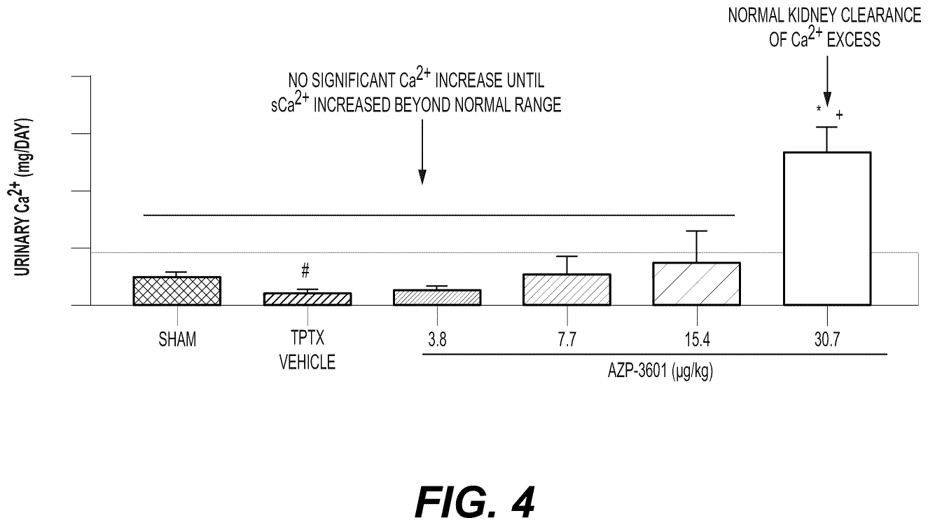

To examine the effect of repeated dosing with AZP-3601 on urinary calcium levels, TPTX rats were treated with daily subcutaneous injection of AZP-3601 with doses ranging from 3.8 to 30.7 μg/kg/day. As additional controls, separate groups of TPTX rats received daily injections of either natural PTH (1-84) or the vehicle, and a group of normal (with intact thyroid and parathyroid glands, or sham) rats were injected with vehicle. Despite the increase in serum calcium induced by AZP-3813, there was no significant increase in urinary calcium except with the highest dose of 30.7 μg/kg/day, which induced overt hypercalcemia. The observed increase in urinary calcium excretion with the high dose was a normal kidney response to clear excess blood calcium (FIG. 4).

### Example 5: Assessing the Biological Activity of AZP-3601 in Non-Human Primates

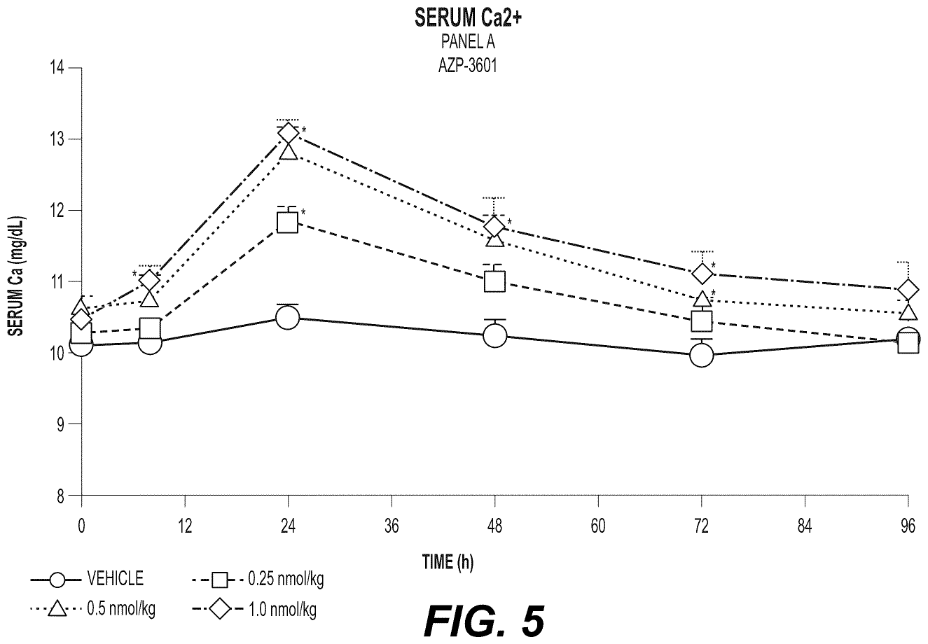

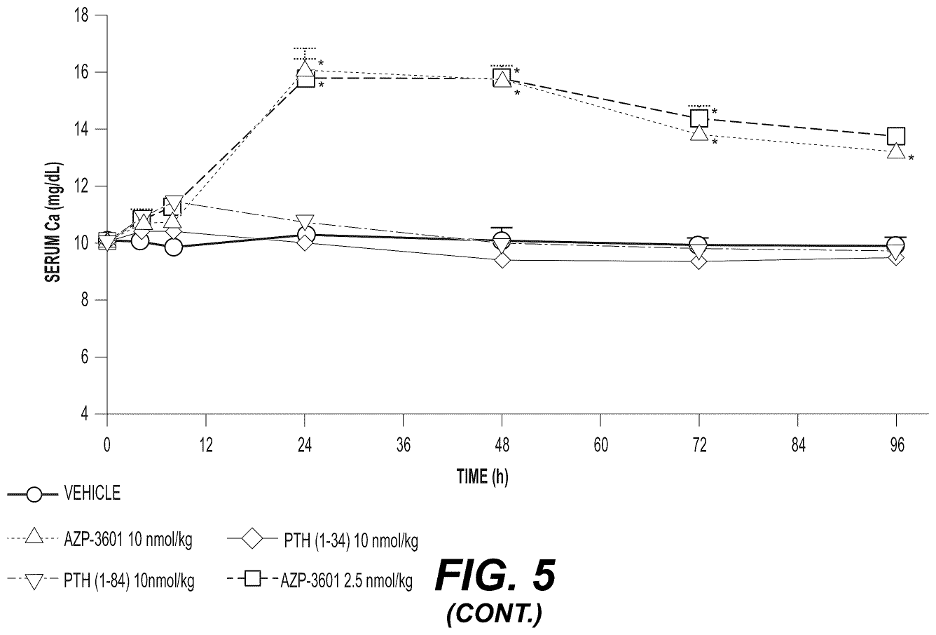

To further examine how the potent and sustained biological activity induced by AZP-3601 could translate into potential clinical benefit for hypothyroid patients, its activity was examined in normal cynomolgus monkeys, a species much closer to humans and the most relevant for first in human dose-selection. Single injections of AZP-3601 were administered to normal cynomolgus monkeys. AZP-3601 was detectable in plasma for only up to one hour post-dosing, while the effect on serum calcium persisted over days. The dissociation between pharmacokinetic profile and pharmacodynamic effect is due to the short circulating half-life of AZP-3601 as opposed to the prolonged signaling caused by AZP-3601's potent interaction with the R⁰ conformation of the PTH1 receptor. AZP-3601 also dose-dependently increased blood calcium levels. As can be seen in panel A of FIG. 5, a significant increase in blood calcium was observed at 24 hours after injection of 1.1 μg AZP-3601/kg (0.25 nmol/kg), and a maximal effect was observed with 2.1 μg/kg (0.5 nmol/kg). With the higher doses of 2.1 and 4.2 μg AZP-3601/kg (0.5 and 1 nmol/kg), a significant elevation of blood calcium levels was observed as early as 12 hours after injection and remained significantly elevated up to 72 hours after the single injection.

In a second study as illustrated in panel B of FIG. 5, monkeys were injected with a single dose of either natural PTH (1-84) at 95 μg/kg, natural PTH (1-34) at 42 μg/kg or AZP-3601 at 42.7 μg/kg, which were equal doses of 10 nmole/kg, adjusted for the molecular weight of the individual compounds. In addition, a lower dose of 10.7 μg AZP-3601/kg (2.5 nmole/kg) was also tested. The two doses of AZP-3601 similarly induced increased blood calcium levels well above the normal range, representing overt hypercalcemia, that remained elevated for at least four days after injection. In contrast, the equivalent doses of the natural PTH compounds, PTH (1-84) and PTH (1-34), only modestly increased blood calcium, and the calcium levels returned to baseline within 24 hours. In these studies, with normal cynomolgus monkeys, AZP-3601 was approximately 40-fold more potent than either natural PTH (1-84) or PTH (1-34) in elevating serum calcium and produced far longer lasting effect (days for AZP-3601 vs. hours for natural PTH).

### Example 6: Direct Comparison of Distal Femur in TPTX Rats Following 14-Day Treatment with Either Daily PTH(1-34) Injection, Continuous PTH(1-34) Infusion or Daily AZP-3601 Injection, at Doses that Normalize Serum Calcium

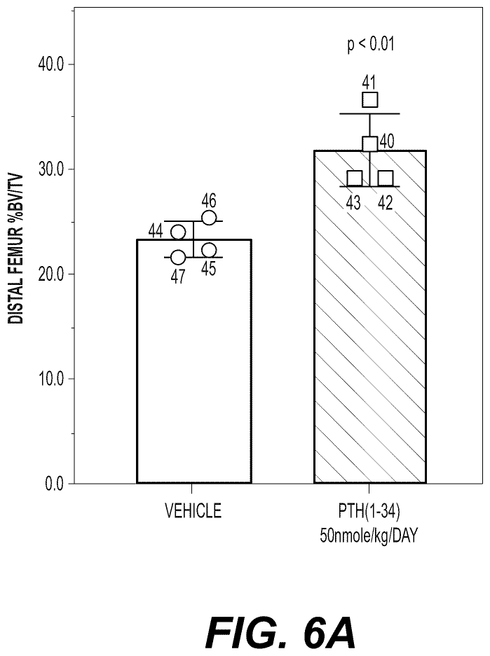

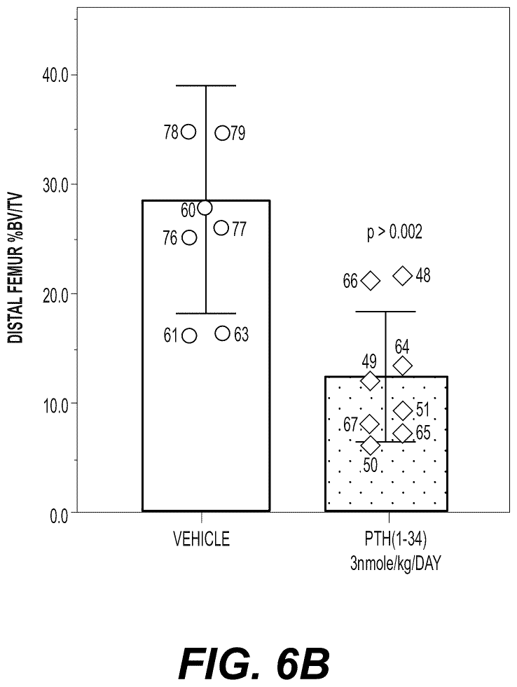

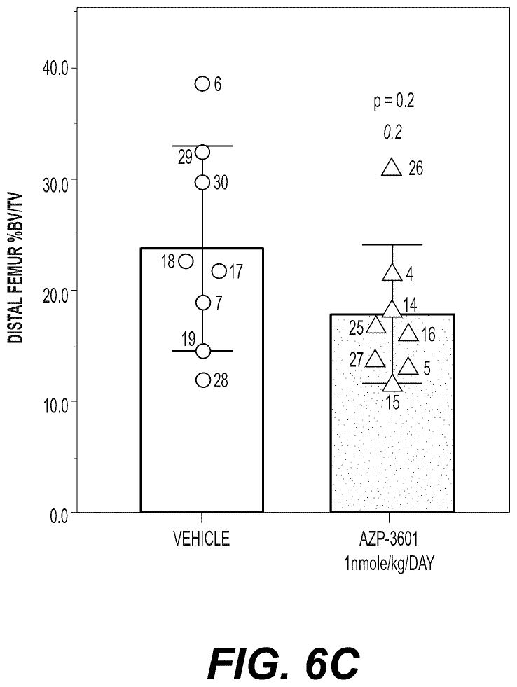

To directly compare the effect of daily subcutaneous injection of AZP-3601 to daily subcutaneous injection and continuous infusion of PTH(1-34), dose levels for each regimen that normalized serum calcium levels in TPTX rats were utilized. These doses corresponded to 50 nmoles/kg/day (205 μg/kg/day) for PTH(1-34) injected daily, 3 nmoles/kg/day (12.3 μg/kg/day) for PTH(1-34) continuously infused, and 1 nmole/kg/day (4.2 μg/kg/day) for AZP-3601 injected daily. After 14 days of treatment, femurs were collected and examined for bone mineral density (BMD) by quantitative computed tomography. A significant increase in BMD was observed with daily injection of PTH(1-34), a significant decrease in BMD was observed with PTH(1-34) continuously infused, and no significant effect on BMD was observed with daily injection of AZP-3601. These results with PTH(1-34) are very much in keeping with previous studies comparing intermittent and continuous administration of natural PTH, and provide evidence of a uniquely neutral effect of AZP-3601, likely the result of a combined effect of its enhanced signaling due to potent interaction with the R⁰ conformation of the PTH1 receptor and its short circulating half-life (FIGS. 6A-6C)

### Example 7: Evaluation of AZP-3601 on Bone Mineral Density in HP TPTX Rat Model

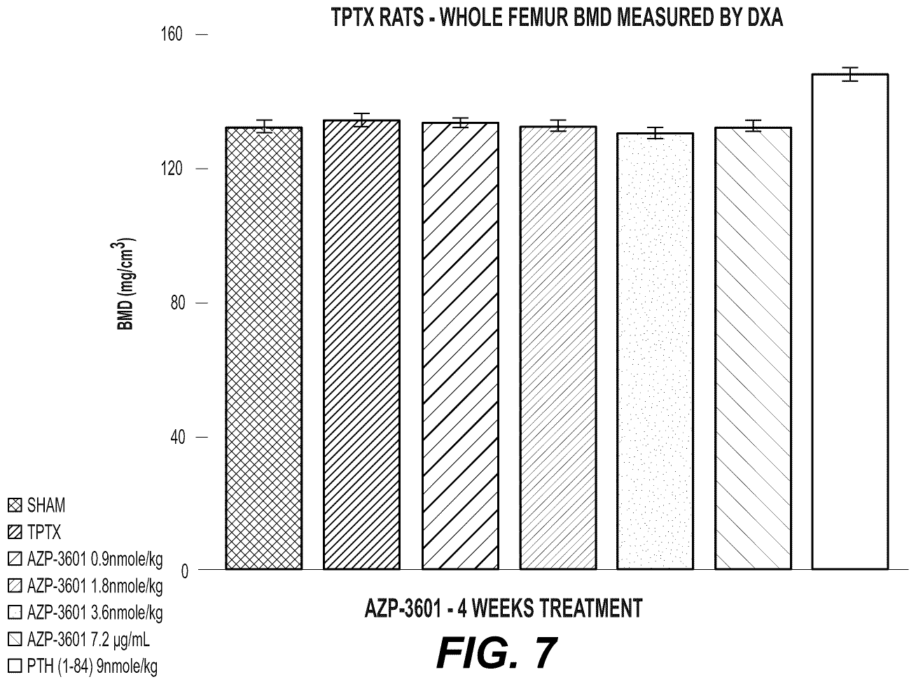

To examine the effect of repeated dosing with AZP-3601, TPTX rats were treated with daily subcutaneous injections of either vehicle or AZP-3601 with doses ranging from 3.8 to 30.7 μg/kg/day (0.9-7.2 nmole/kg/day). As additional controls, a separate group of TPTX rats received daily injections of natural PTH (1-84), and a group of normal (with intact thyroid and parathyroid glands, or sham) rats were injected with vehicle. The results of this study are presented in FIG. 7. The chronic treatment with AZP-3601 was shown to have no impact on the bone. Both whole femurs and lumbar spine were analyzed using a technique called dual-energy x-ray absorptionametry, or DXA, which measures BMD. This analysis revealed no significant effects of AZP-3601 treatment, as compared with bones from animals treated with vehicle alone. In contrast, the TPTX-rats injected once daily with natural PTH (1-84) showed a statistically significant increase in BMD in both lumbar spine and whole femur (p<0.05). The AZP-3601 treatment groups showed no significant differences in bone structure using the technique of micro-computerized tomography.

In contrast, in a reported study (Holten-Andersen et al. JBMR 34:2075, 2019, Ref. 20) also using TPTX rats treated for a similar length of time with the TransCon® formulation of natural PTH (1-34), which produces a continuous, non-pulsatile, infusion-like pharmacokinetic profile, a marked decrease in BMD, compared to both the sham-operated and TPTX rats treated with the vehicle alone, was observed. Prior third-party studies similarly have demonstrated that intermittent administration of natural PTH increased BMD, while continuous, non-pulsatile infusion resulted in decreased BMD. The impact of continuous, non-pulsatile exposure to natural PTH compounds on the bone can also clearly be observed with certain pathologies such as hypercalcemia of malignancy where continuous, non-pulsatile production of PTH-related peptide, or PTHrP, which also acts on the PTH1 receptor, induces highly significant loss of BMD in a matter of months.

### Example 8: AZP-3601 has No Impact on Bone Parameters Following Chronic Treatment of Non-Human Primates

To further examine the effect of chronic AZP-3601 treatment on bone, both 13-week and 39-week studies were conducted in non-human primates (NHP), which are considered a relevant species with regard to AZP-3601 effects on serum calcium. Groups of 3 or 4 Cynomolgus monkeys of each sex were given daily subcutaneous injections of either vehicle or AZP-3601 at doses of 1, 2.5 or 10 μg/kg for either 13 or 39 weeks, as outlined in Table 2. The doses selected are either within or significantly exceed the anticipated therapeutic dose range for cHP patients.

TABLE 2
TREATMENT OF NON-HUMAN PRIMATES WITH AZP-3601

| | | Number of animals | |
|---|---|---|---|
| | Dose | (males + females) | |
| | (μg/kg/day) | 13-week study | 39-week study |
| Control (saline) | — | 3 + 3 | 6 + 6 |
| Low dose | 1 (22 mg/day)* | 3 + 3 | 4 + 4 |
| Mild dose | 2.5 (55 mg/day)* | 3 + 3 | 4 + 4 |
| High dose | 10 (220 mg/day)* | 3 + 3 | 6 + 6 |

*Human equivalent - dose for 70 kg individual

### 13-Week Study:

Ex-vivo bone mineral density (BMD) by dual-energy X-ray absorptiometry (DXA) at week 13;
Histopathological examination of bone at week 13.

### 39-Week Study:

Serum bone biomarkers at baseline and weeks 4, 8, 13, 26 and 39;
In-life BMD by quantitative computed tomography (qCT) at baseline and weeks 26 and 39;
Histopathological examination of bone at week 39.

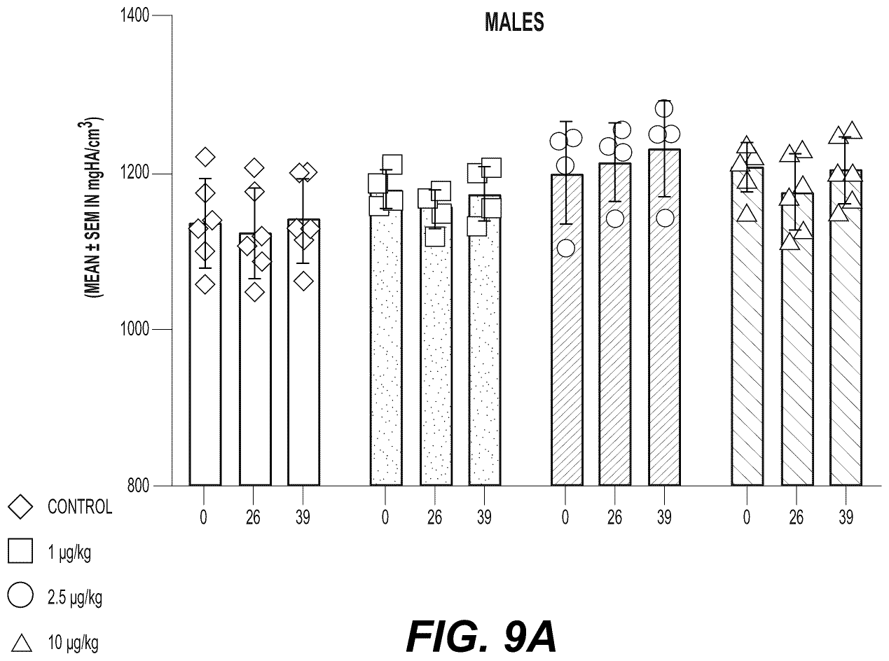

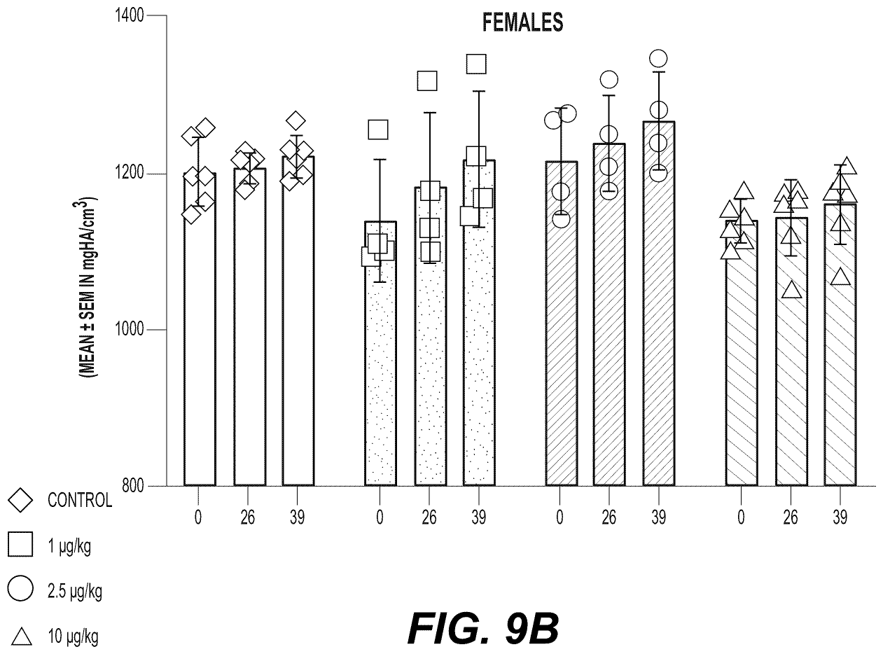

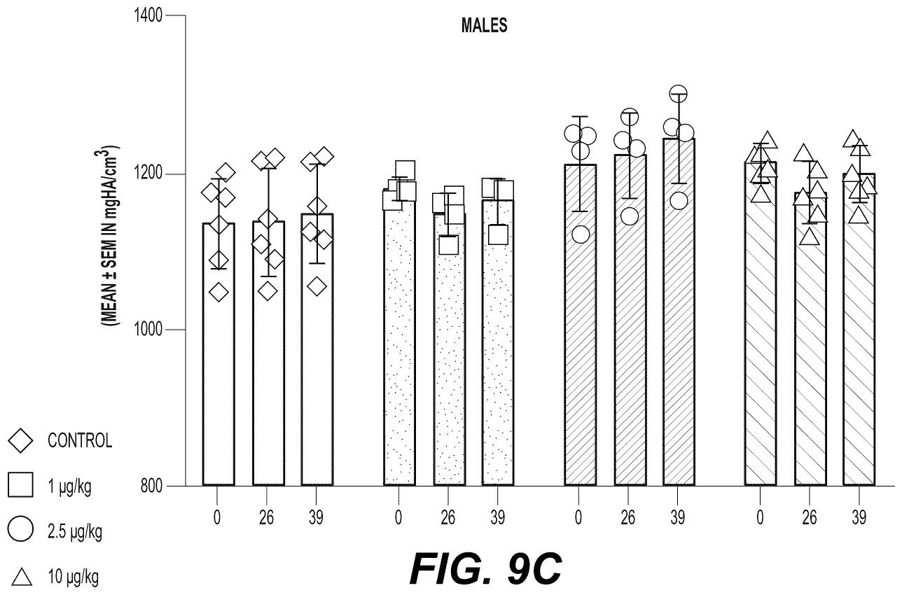

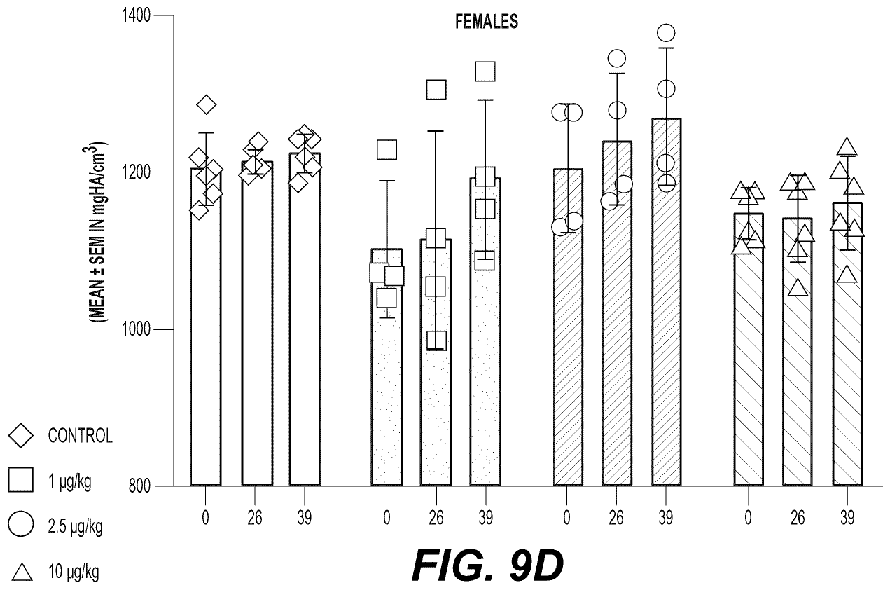

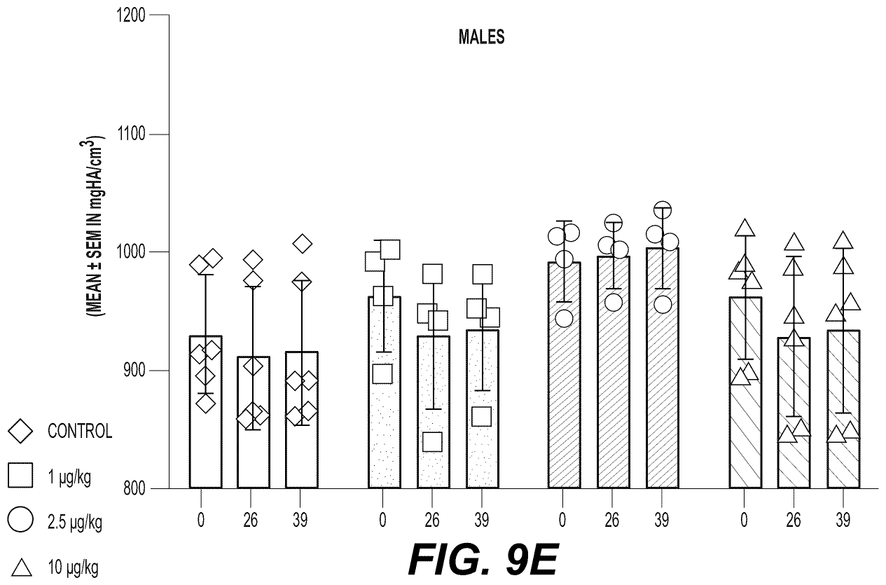

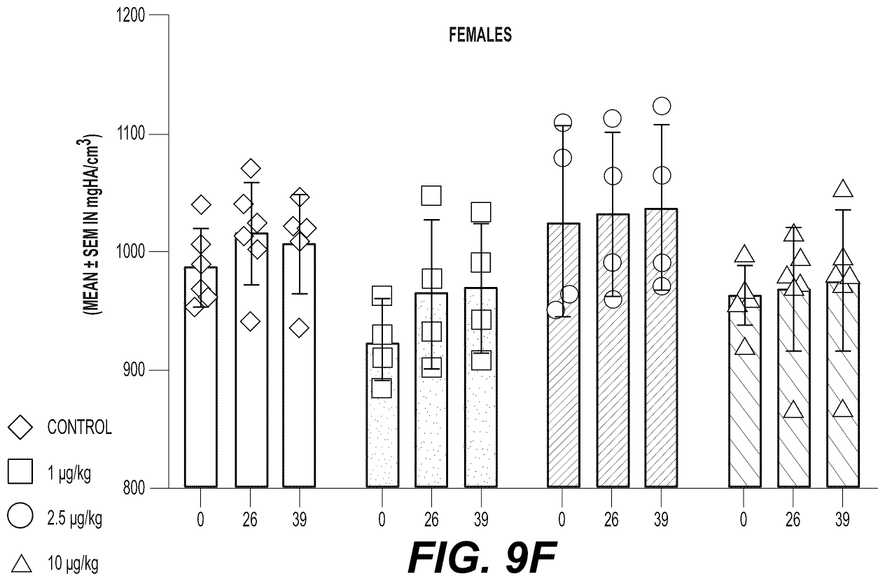

At the end of the 13-week study, right femur, right tibia and the L4 lumbar vertebra were collected from 3 animals/group/sex and bone mineral density (BMD) was measured by dual-energy X-ray absorptiometry. The left femur was submitted to histopathological examination. There was no evidence of any treatment-related effect on either BMD or histopathology. In the 39-week study, blood samples were collected from 4 animals/group/sex prior to and during weeks 4, 8, 13, 26 and 39 of treatment for measurement of bone biomarkers. In-life BMD of left femur, left tibia and L4 lumbar vertebra was measured by quantitative computed tomography (qCT) prior to and during weeks 26 and 39 of treatment (FIGS. 9A, 9B, 9C, 9D, 9E and 9F). At the end of the treatment period, femurs were processed for histopathological examination.

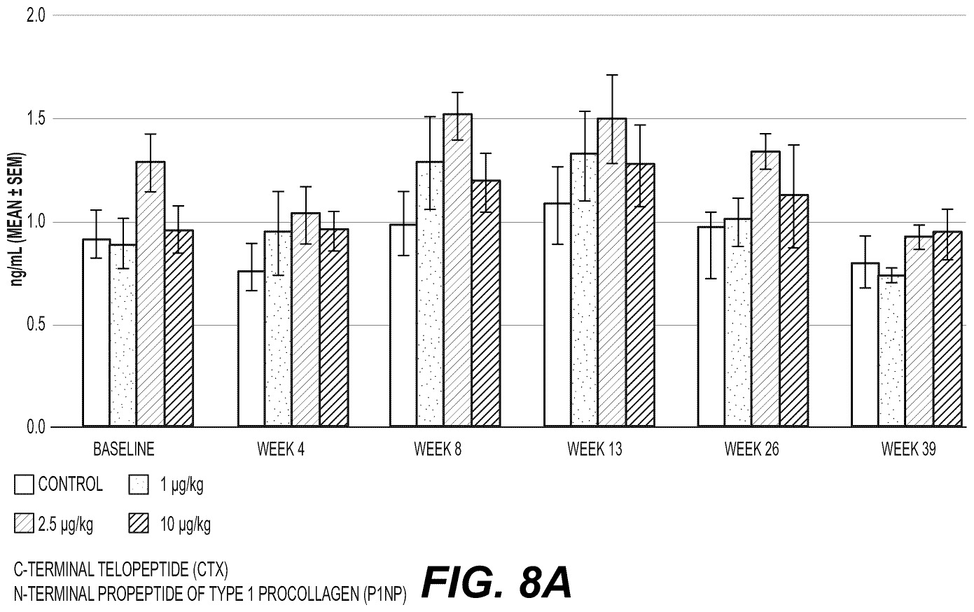

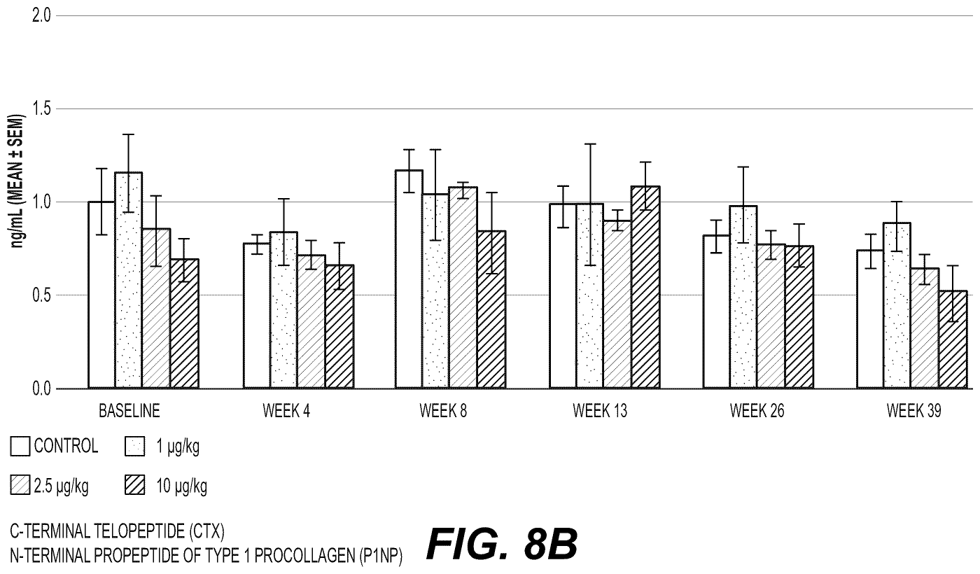

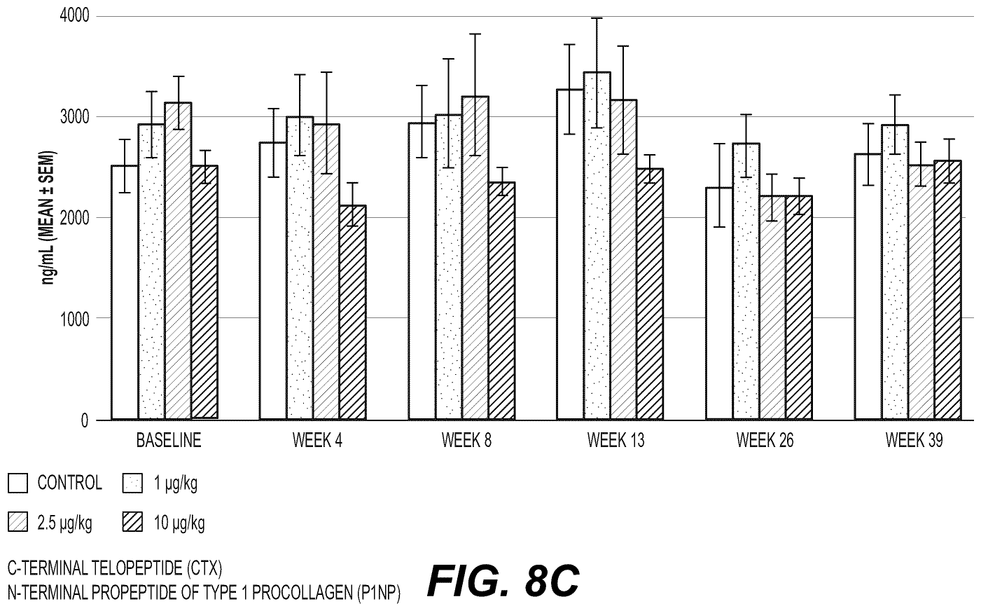

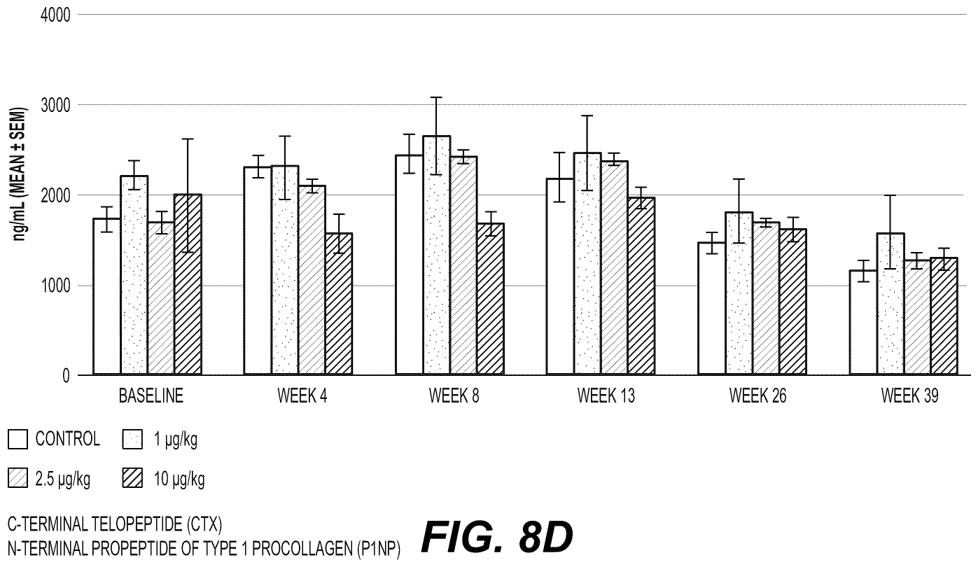

Analysis of blood samples for the anabolic bone biomarker, N-terminal propeptide of type 1 procollagen (P1NP), and the catabolic bone biomarker, C-terminal telopeptide (CTx), revealed no treatment-related changes at any time in either sex (FIGS. 8A, 8B, 8C and 8D). Prior to treatment, BMD was homogeneous among all groups for all the three bone sites examined. Over the course of the study, there were no statistically significant changes in BMD as compared to pre-treatment values, regardless of sex or AZP-3601 treatment. Subsequent histological examination of the femur revealed no noteworthy findings.

It is believed that this apparent neutral effect of AZP-3601 on bone is unique and likely stems from its potent binding to the R⁰ conformation of the PTH1 receptor, its short circulating half-life and its greatly enhanced and prolonged biological effect that allows use of a substantially lower dose.

In HP patients, there is a precarious balance between the potentially protective effect of increased BMD against patients' profoundly abnormal bone microarchitecture, which translates to a neutral risk of fracture, as observed in population studies. However, any treatment or condition that decreases BMD has the potential to shift the balance in favor of the abnormal microarchitecture and could increase the risk of fracture. Consequently, a treatment with a neutral effect on bone can be seen as a substantial advantage in this patient population.

The results presented in Examples 6, 7 and 8 demonstrate an absence of deleterious effect of chronic AZP-3601 treatment on bone, further substantiating the potential of AZP-3601 as a treatment for HP that does not compromise bone integrity.

### Example 9: Assessment of Safety and Tolerability of PTH Compound Following Single and 2-Week Multiple Ascending Doses Administered by Subcutaneous (Sc) Injection in Healthy Subjects

Part A: this part of the assessment was a randomized, double-blind, placebo-controlled single ascending dose (SAD) study in healthy male subjects to evaluate the safety and tolerability, PK, and PD of a PTH compound having SEQ ID NO: 10 (AZP-3601). Subjects were screened between 28 days and 3 days prior to study drug administration. Up to 7 sequential cohorts were planned for this part of the study. The first cohort included 4 subjects of which 3 subjects were randomized to receive AZP-3601 and 1 subject was randomized to receive placebo. The subsequent cohorts included 8 subjects each (6 receiving AZP-3601 and 2 receiving placebo in a randomized and double-blind manner). Subjects in Part A received the study drug at the clinical site as a single sc abdominal injection in the morning. Actual doses administered in the SAD part in healthy volunteers are presented below:
Cohort A1: single sc dose of 5 mg AZP-3601 (n=3) or matching placebo (n=1) on Day 1
Cohort A2: single sc dose of 10 mg AZP-3601 (n=6) or matching placebo (n=2) on Day 1
Cohort A3: single sc dose of 20 mg AZP-3601 (n=6) or matching placebo (n=2) on Day 1
Cohort A4: single sc dose of 40 mg AZP-3601 (n=6) or matching placebo (n=2) on Day 1
Cohort A5: single sc dose of 60 mg AZP-3601 (n=6) or matching placebo (n=2) on Day 1
Cohort A6: single sc dose of 120 mg AZP-3601 (n=6) or matching placebo (n=2) on Day 1
Cohort A7: single sc dose of 90 mg AZP-3601 (n=6) or matching placebo (n=2) on Day 1.

A median albumin-corrected peak serum calcium level of ≥10.5 mg/dL (2.6 mmol/L), as based on blinded data (AZP-3601-treated subjects only) or an albumin-corrected peak serum calcium level of ≥12 mg/dL (3.0 mmol/L) of at least 1 subject were used as a general guideline to perform smaller dose escalations. Within each cohort, subjects were sequentially dosed as follows: Two sentinel subjects were dosed on the first dosing day (1 subject receiving AZP-3601 and 1 subject receiving placebo). Serum calcium was monitored until levels were comparable to baseline values and/or within the normal range. There was an interval of at least 7 days between the last dose in one cohort and the first dose in the next cohort. Each subject participated in only 1 cohort during the study.

Part B: this part of the assessment was a randomized, double-blind, placebo-controlled multiple ascending dose (MAD) study in healthy male or female subjects of non-childbearing potential to evaluate the safety and tolerability, PK, and PD of AZP-3601. Subjects were screened between 28 days and 3 days prior to (the first) study drug administration. Four sequential cohorts were planned for this part of the study. Each cohort included 10 subjects (8 receiving AZP-3601 and 2 receiving placebo in a randomized manner). Subjects in Part B received the study drug at the clinical site for 14 days as daily sc abdominal injections (with rotation of injection sites every dosing day) in the morning. Actual doses administered in the MAD part in healthy volunteers are presented below:
Cohort B1: multiple sc doses of 10 mg AZP-3601 (n=8) or matching placebo (n=2) once daily (qd) from Day 1 to Day 14
Cohort B2: multiple sc doses of 20 mg AZP-3601 (n=8) or matching placebo (n=2) qd from Day 1 to Day 14
Cohort B3: multiple sc doses of 40 mg AZP-3601 (n=8) or matching placebo (n=2) qd from Day 1 to Day 14
Cohort B4: multiple sc doses of 60 mg AZP-3601 (n=8) or matching placebo (n=2) qd from Day 1 to Day 14
Cohort B5: multiple sc doses of 80 mg AZP-3601 (n=8) or matching placebo (n=2) qd from Day 1 to Day 14

The subjects received the study drug qd for a total duration of 14 days in order to obtain a steady state of PD parameters, including serum and urinary calcium. Serum calcium was monitored until levels were comparable to baseline values and/or within the normal range. Part B commenced after the first 3 cohorts of Part A had been completed. A median albumin-corrected peak serum calcium level of ≥10.5 mg/dL (2.6 mmol/L), as based on blinded data (AZP-3601-treated subjects only) or an albumin-corrected peak serum calcium level of ≥12 mg/dL (3.0 mmol/L) of at least 1 subject were used.

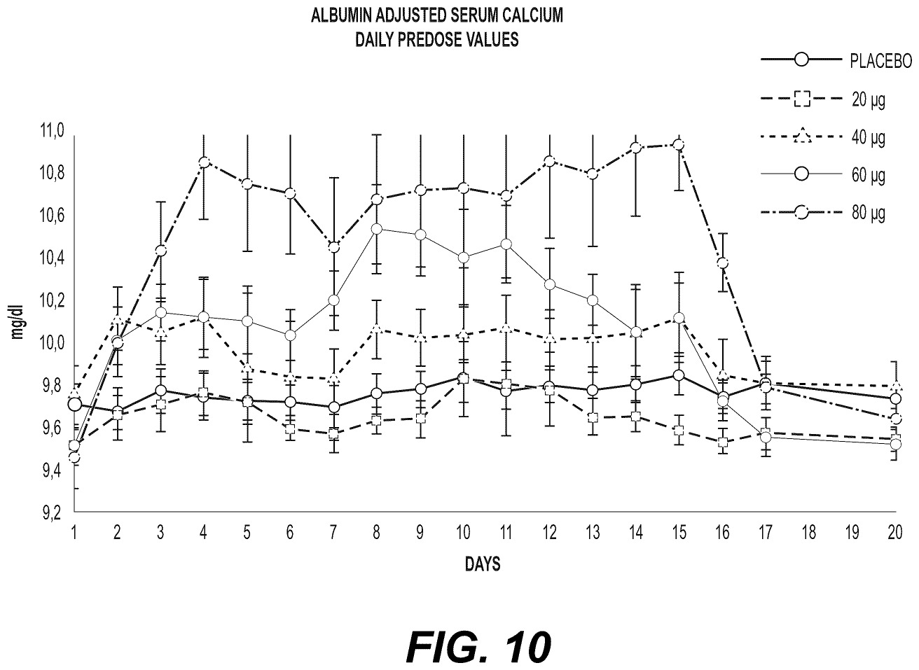

FIG. 10 shows that as compared with placebo controls, AZP-3601 treatment produced a clear, dose-dependent increase in mean albumin-adjusted serum calcium values from baseline. The normal physiological diurnal variation of albumin-adjusted serum calcium was gradually attenuated with 5 μg and 10 μg AZP-3601, and was completely eliminated with 20 μg. With the dose of 40 μg AZP-3601, mean albumin-adjusted serum calcium values were significantly increased but stayed within normal laboratory range and remained elevated through at least 24 hours post-administration. A dose-dependent decrease in mean endogenous serum PTH was observed which was significantly correlated with the concomitant increase in mean serum calcium. These data provide evidence of the pharmacodynamic effect of AZP-3601 in healthy humans characterized by a sustained calcemic response for at least 24 hours following a single administration.

### Example 10: Assessment of Safety and Tolerability of PTH Compound Following Single and 4-Week Multiple Ascending Doses Administered by Sc Injection in Subjects with HP

Part C: Part C of the assessment was an open-label multiple ascending dose (MAD) study in male or female subjects with hypoparathyroidism (HP) who are on standard or care treatment (treatment with oral calcium and active vitamin D) to evaluate the safety and tolerability, PK, and PD of AZP-3601. Up to 2 cohorts of approximately 12 patients each were planned for this part of the study. Prior to the treatment period, there was an optimization period of up to 8 weeks during which doses of oral calcium and active vitamin D were adjusted to achieve a baseline target range of albumin-corrected serum calcium (7.8 to 9.0 mg/dL, ie, 1.95 to 2.25 mmol/L) and therefore ensuring a close baseline for all patients. During this optimization period, any serum 25-hydroxy vitamin D (native vitamin D) and/or magnesium deficiencies were corrected. During the treatment period, patients in Part C received AZP-3601 for 28 days as daily sc abdominal injections (with rotation of injection sites every dosing day). Doses of oral calcium and active vitamin D supplements were reduced during the initial 14 days of treatment while maintaining albumin-corrected serum calcium in the target range (7.8 to 9.0 mg/dL, ie, 1.95 to 2.25 mmol/L). Reductions of both oral calcium and active vitamin D supplements were performed using a staged approach until the active vitamin D dose were eliminated and the oral calcium dose were reduced to or below 500 mg/day. Based on animal data, serum calcium was expected to increase progressively following repeated administration of AZP-3601 and PD steady state was expected to be observed within the first 5 days of dosing. Staggered reductions of doses of oral calcium and active vitamin D supplements were therefore performed at near-steady state based on the predose albumin-corrected serum calcium value. The following treatments are administered in Part C in an open-label fashion:
Cohort C1: multiple sc doses of 20 mg AZP-3601 (n=12) qd from Day 1 to Day 28 (dose increase of AZP-3601 to 40 mg was allowed from Day 14 onwards)
Cohort C2: multiple sc doses of a fixed dose of 10 mg AZP-3601 (n=12) qd from Day 1 to Day 28 (dose of AZP-3601 may be increased at a fixed dose of 20 mg from Day 14 onwards)

During the extension phase, patients had their AZP-3601 dose increased in increments of 10 mg to a maximum dose of 60 mg (Cohort 1) and of 80 mg (Cohort 2).

### Example 11: Assessment of Safety and Tolerability of PTH Compound During a 2-Month Treatment Extension Period in Subjects with HP

The extension phase started immediately following Day 28 visit of the Main treatment period. Patients received AZP-3601 for 56 days (2 months) as daily sc injections from Day 29 onwards (with rotation of injection sites each day of administration). The goal was to optimize AZP-3601 dosing across a dose range while doses of oral calcium and active vitamin D were as low as safely possible and albumin-corrected serum calcium was maintained within the target range of 7.8 to 9.0 mg/dL (1.95 to 2.25 mmol/L). For patients who were taking minimal or no supplemental calcium (≤500 mg/day) and no vitamin D on Day 28, the AZP-3601 dose from the previous 14 days of treatment (Part C, Main Treatment Period) was maintained and adjusted when needed during the Extension Phase. For patients who were still taking active vitamin D and/or more than 500 mg/day of oral calcium on Day 28, a progressive reduction of supplements was carried out while increasing the dose of AZP-3601. On Day 29 onwards, patients continued on the same dose of AZP-3601 as on Day 28, then individual titration of AZP-3601 started as early as Day 30 and patients had their dose of AZP-3601 adjusted at any time during the Extension Phase. Patients could have their AZP-3601 dose increased, as previously defined, with the goal of achieving or maintaining albumin-corrected serum calcium in the target range of 7.8 to 9.0 mg/dL (1.95 to 2.25 mmol/L). The AZP-3601 dose was adjusted downward at anytime as needed to maintain albumin-corrected serum calcium within the target range or for any safety concerns. Once patients achieve a stable albumin-corrected serum calcium with the minimum doses of supplements, they were maintained at that dose of AZP-3601.

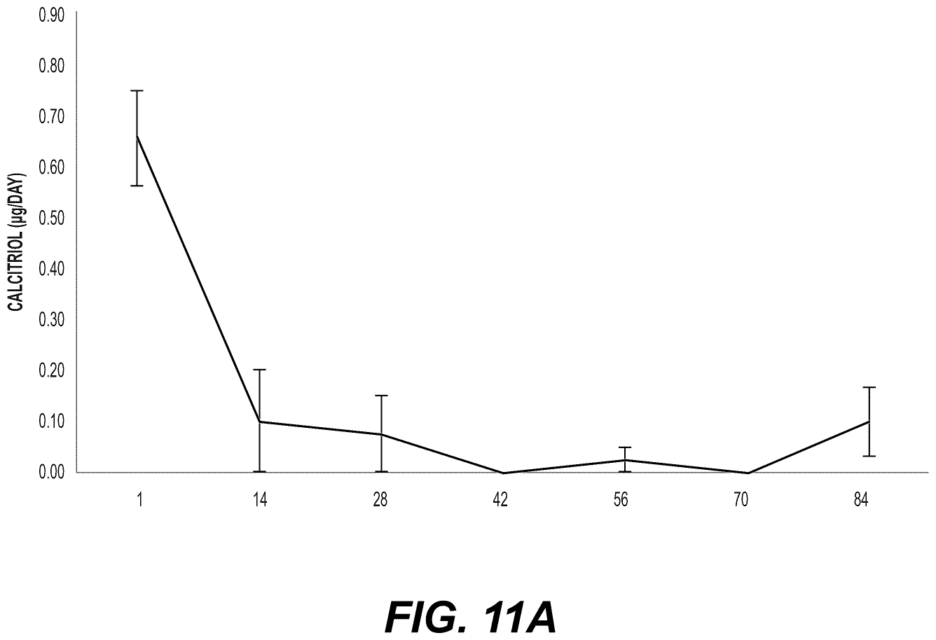

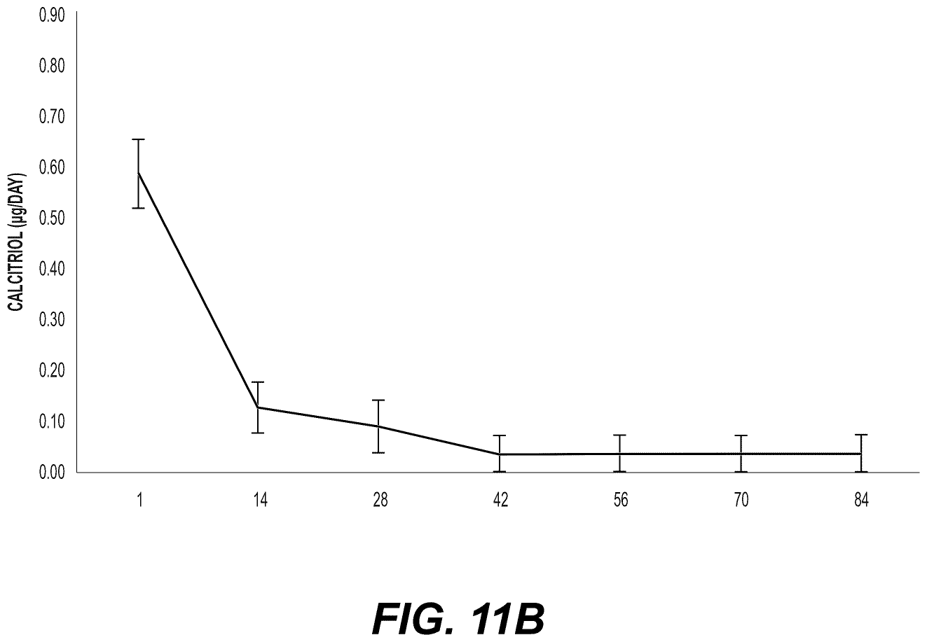

FIGS. 11A and 11B show that administration of AZP-3601 at a staring dose of 20 μg/day (FIG. 11A) and at a starting dose of 10 μg/day (FIG. 11B) in subjects who completed the extension period enabled discontinuation of administration of active vitamin D/calcitriol within 2 weeks of the initiation of the treatment.

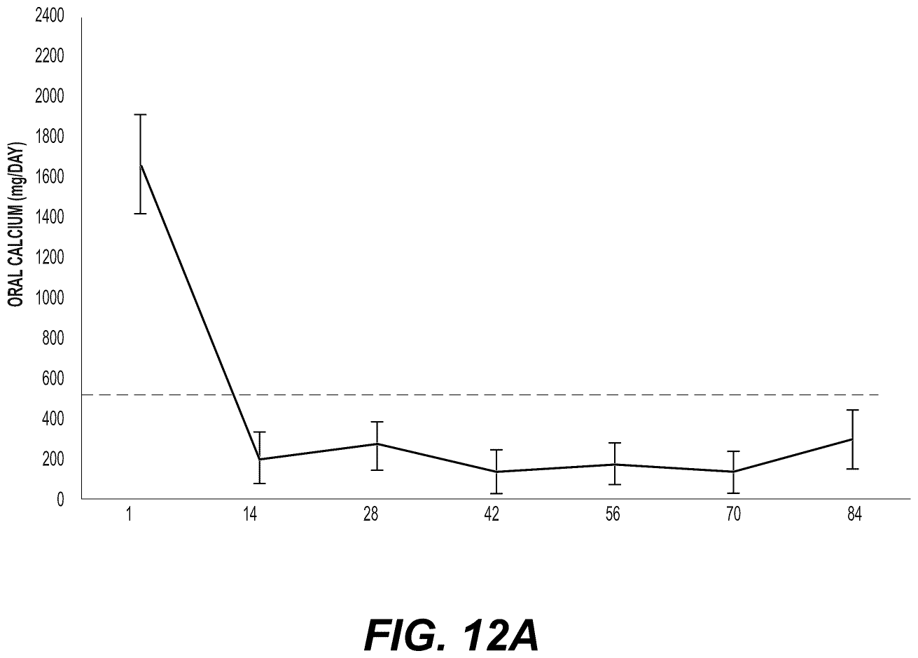

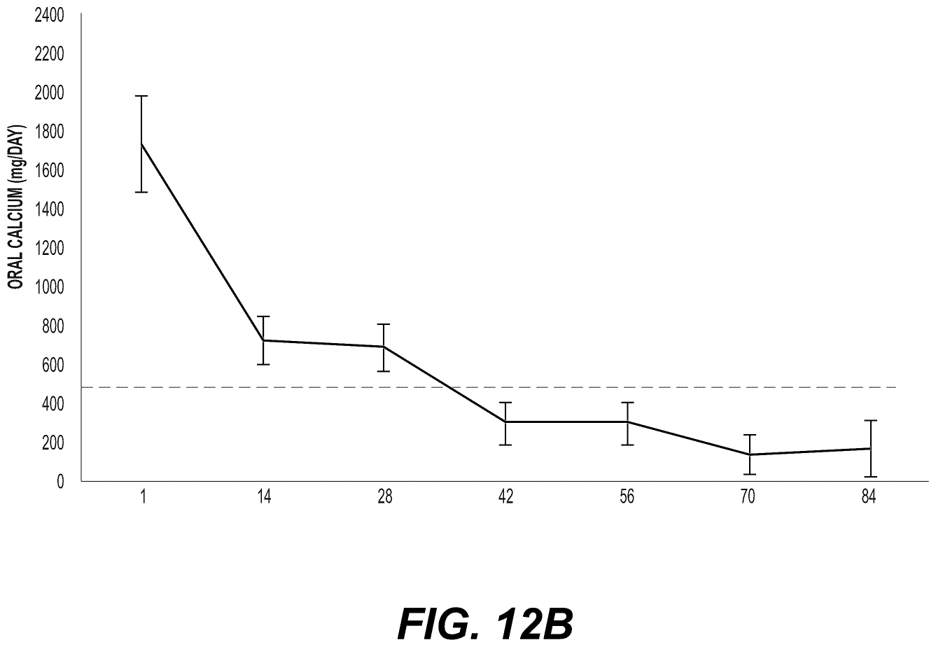

FIGS. 12A and 12B show that administration of AZP-3601 at a starting dose of 20 μg/day (FIG. 12A) and at a starting dose of 10 μg/day (FIG. 12B) in subjects who completed the extension period enabled sustained reduction in oral calcium supplementation below 500 mg/day (dotted line). In Cohort 2, discontinuation of oral calcium supplementation was delayed and required up-titration due to the lower starting dose, supporting a dose-relating effect.

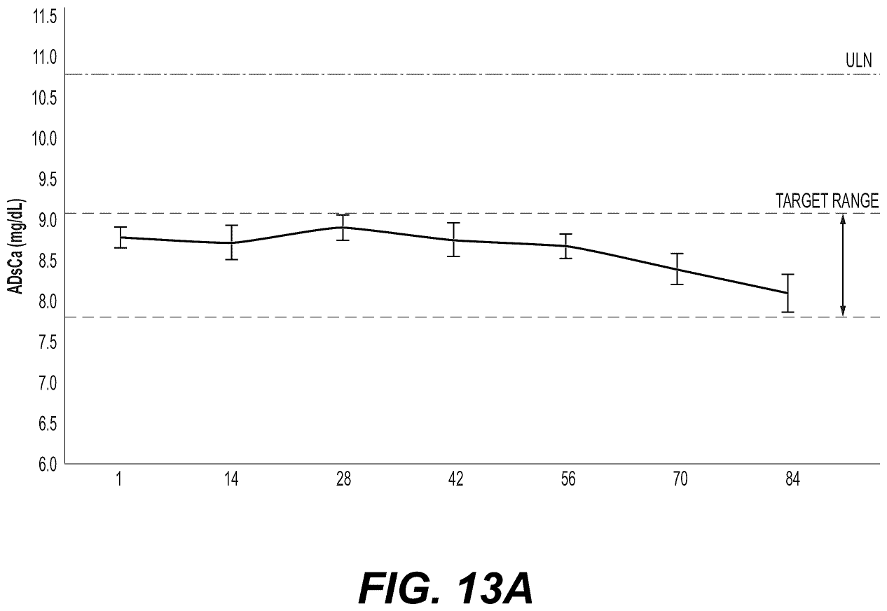

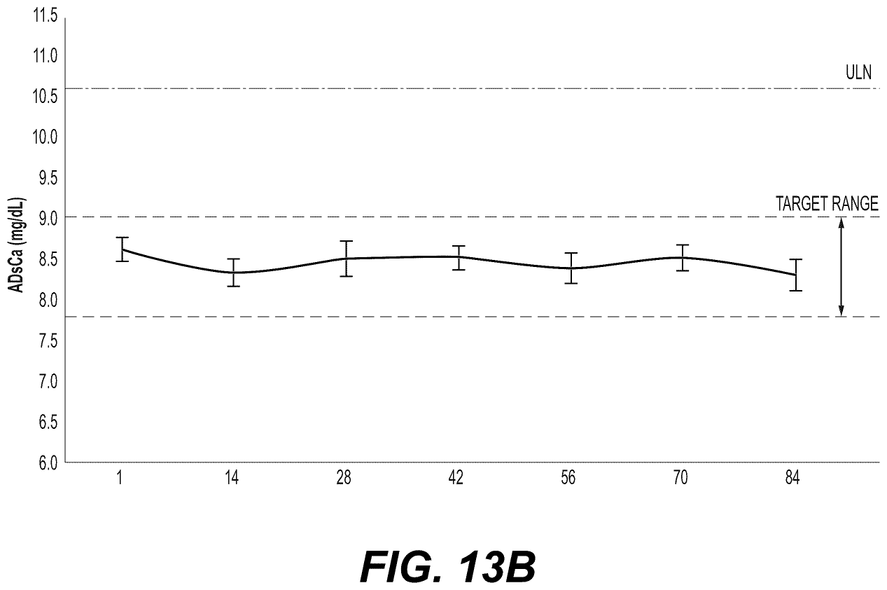

FIGS. 13A and 13B show that administration of AZP-3601 at a starting dose of 20 μg/day (FIG. 13A) and at a starting dose of 10 μg/day (FIG. 13B) in subjects who completed the extension period maintained mean serum calcium within the target range through the 84-day study. These results demonstrate that administration of AZP-3601 allows for rapid discontinuation of the standard of care for HP.

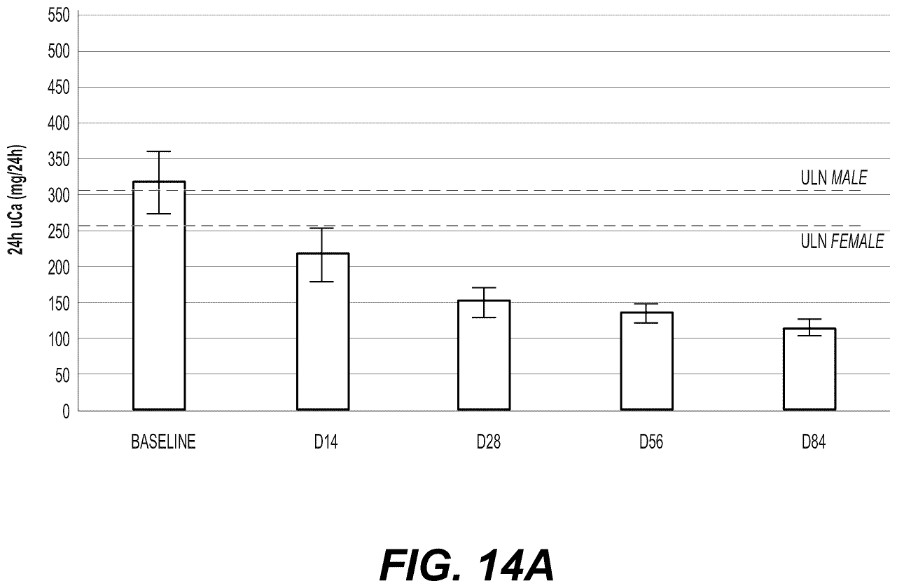

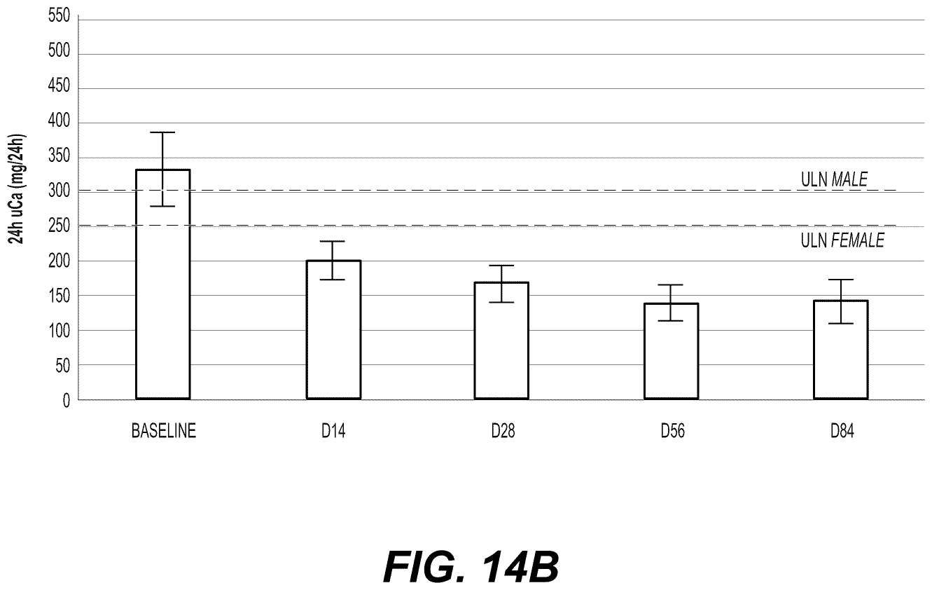

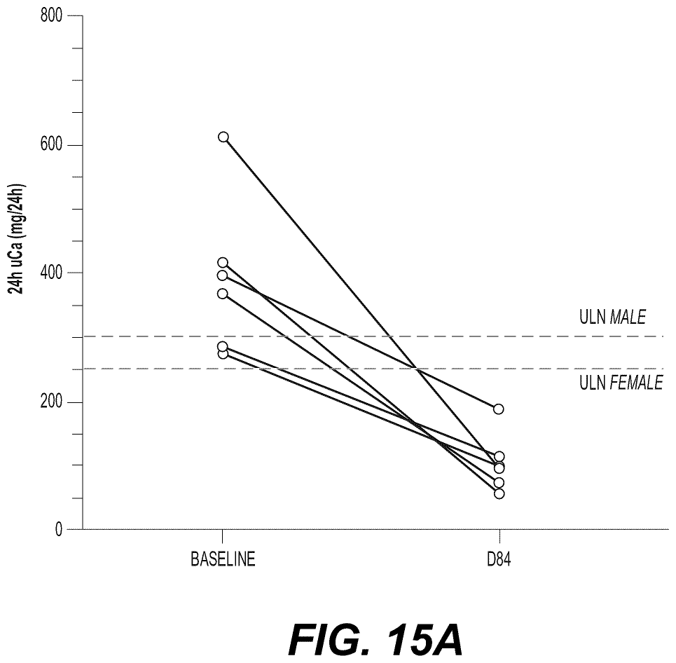

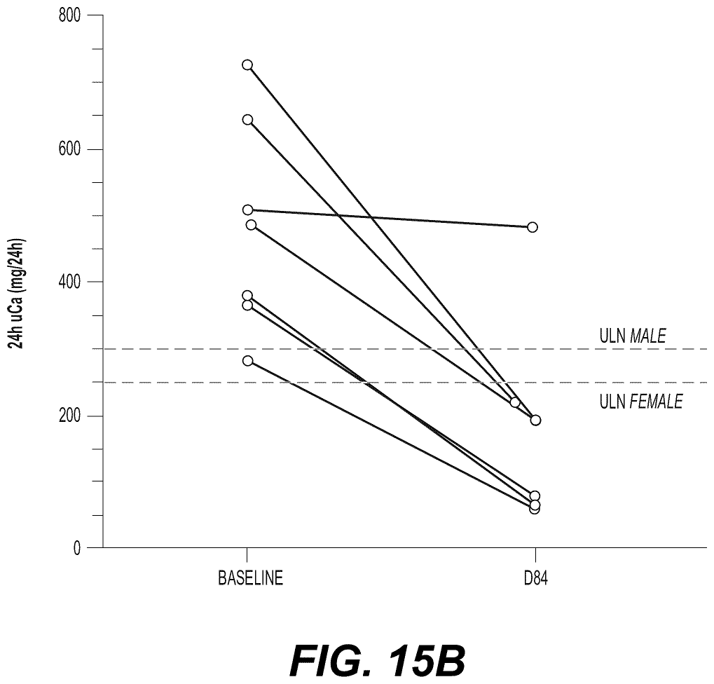

FIGS. 14A and 14B show that administration of AZP-3601, in both cohorts, induced a rapid, prolonged and sustained reduction and normalization in mean 24 h urinary calcium throughout the study treatment duration. The results presented in FIGS. 15A and 15B show that in 12 out of 13 subjects with elevated urinary calcium art baseline, administration of AZP-3601 induced a rapid, profound, and sustained normalization of 24-hour urine calcium in both cohorts.

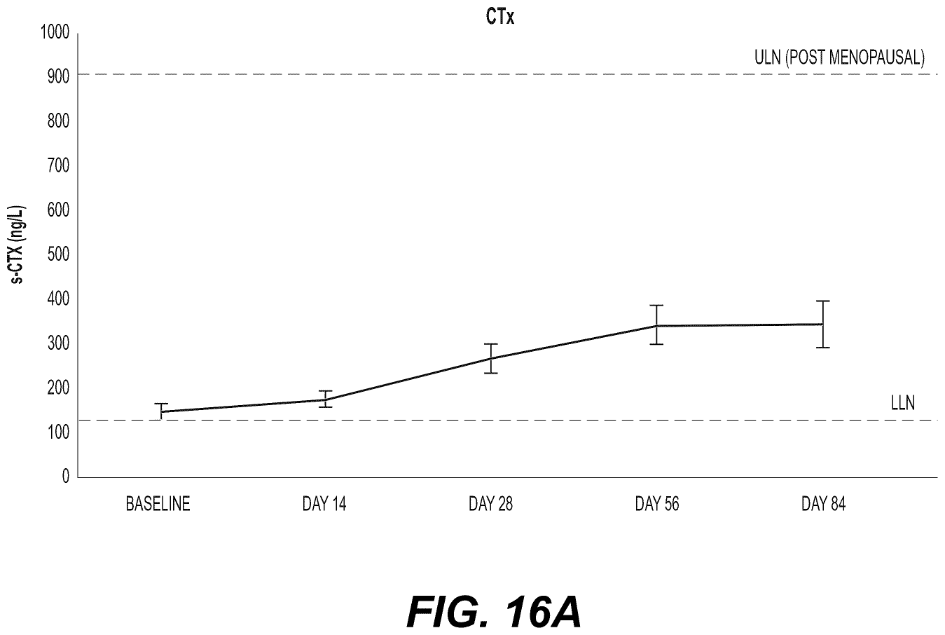

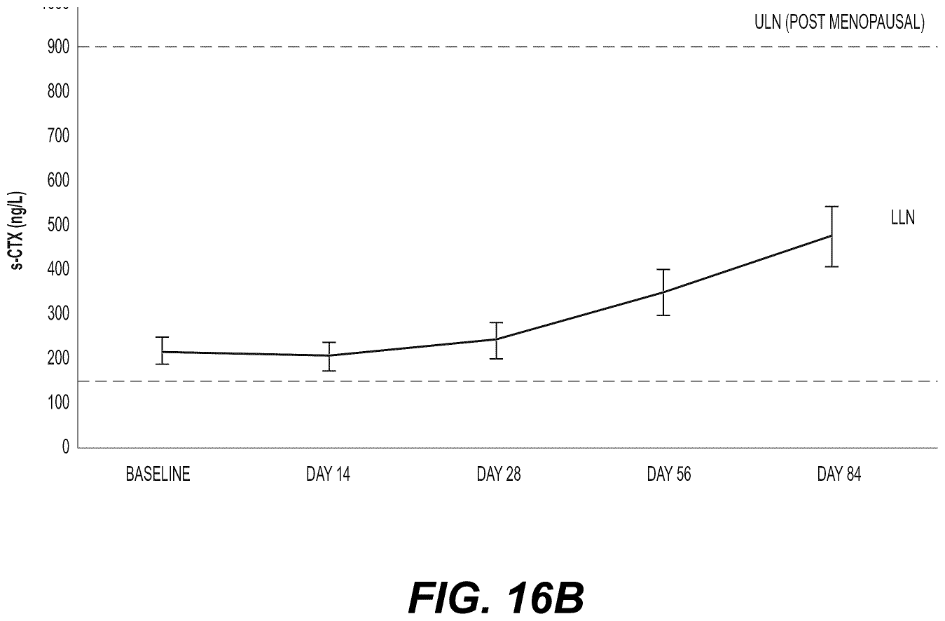

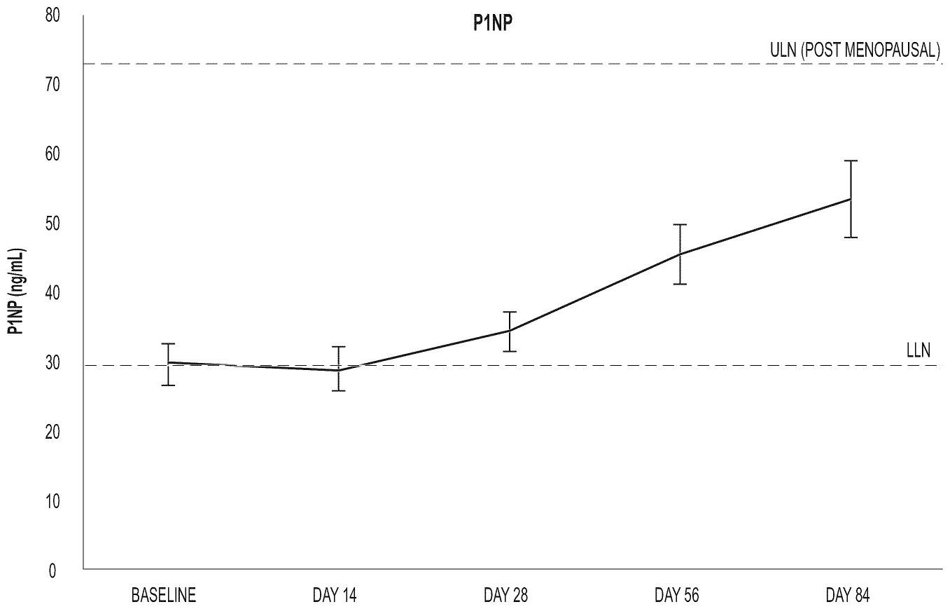

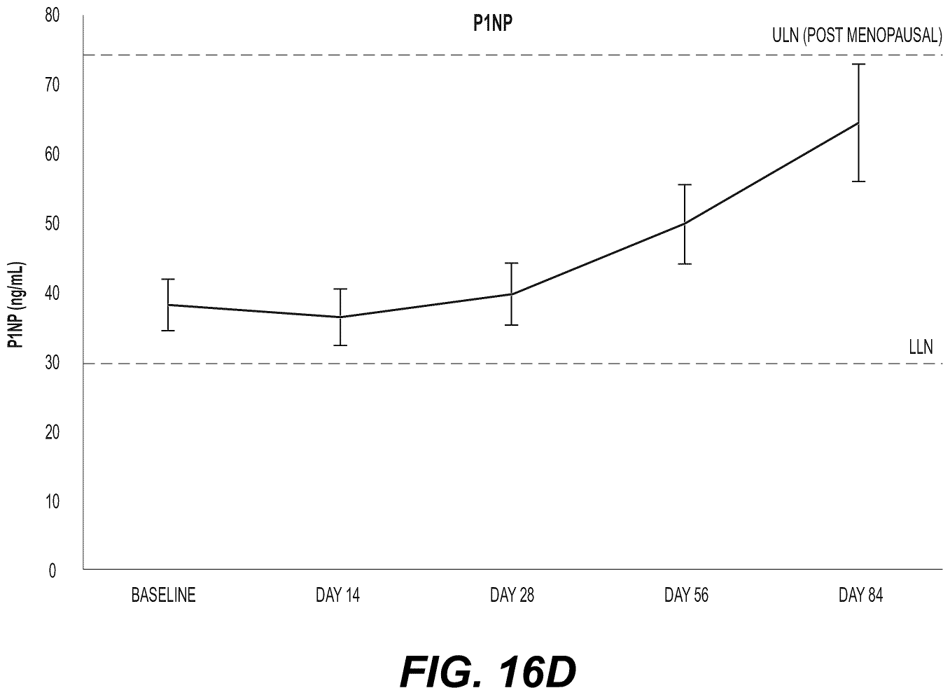

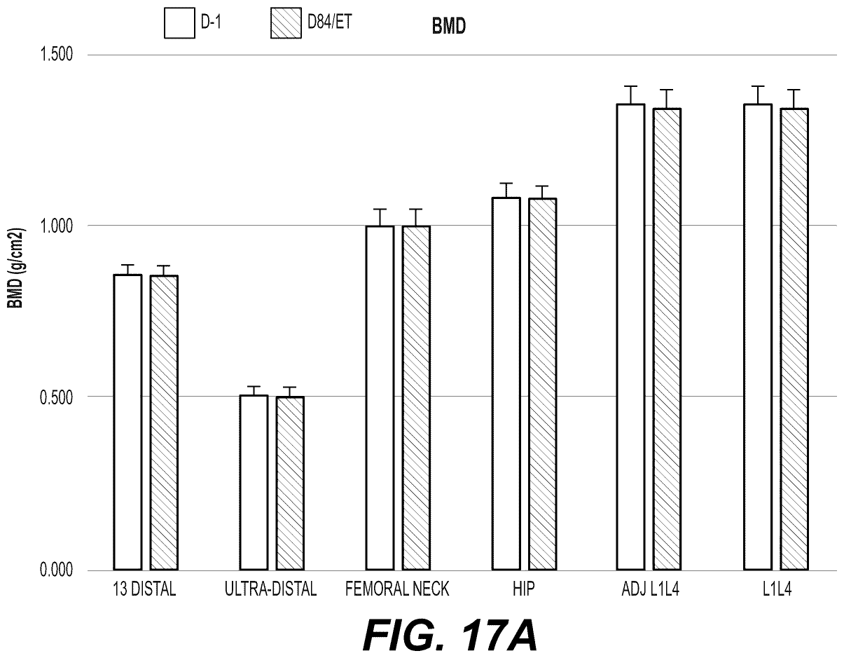

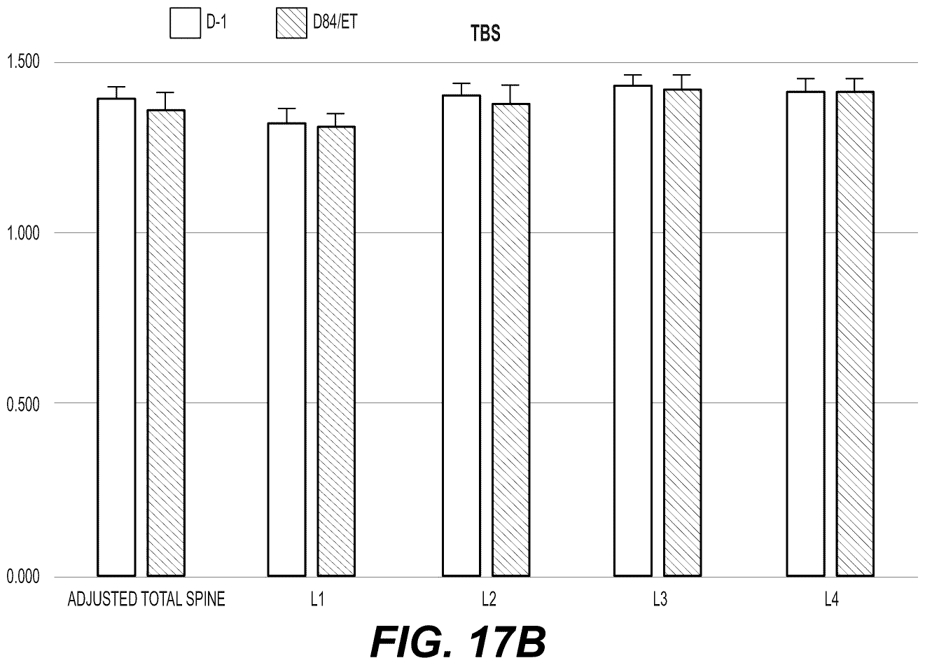

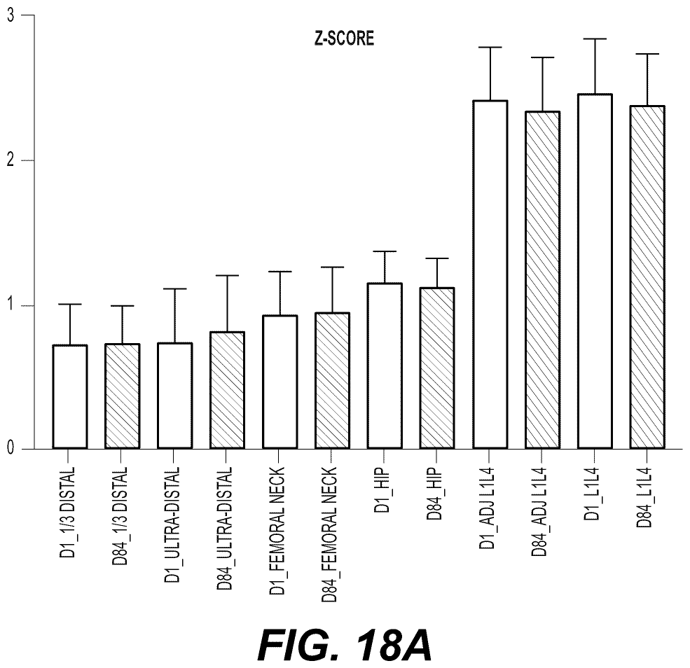

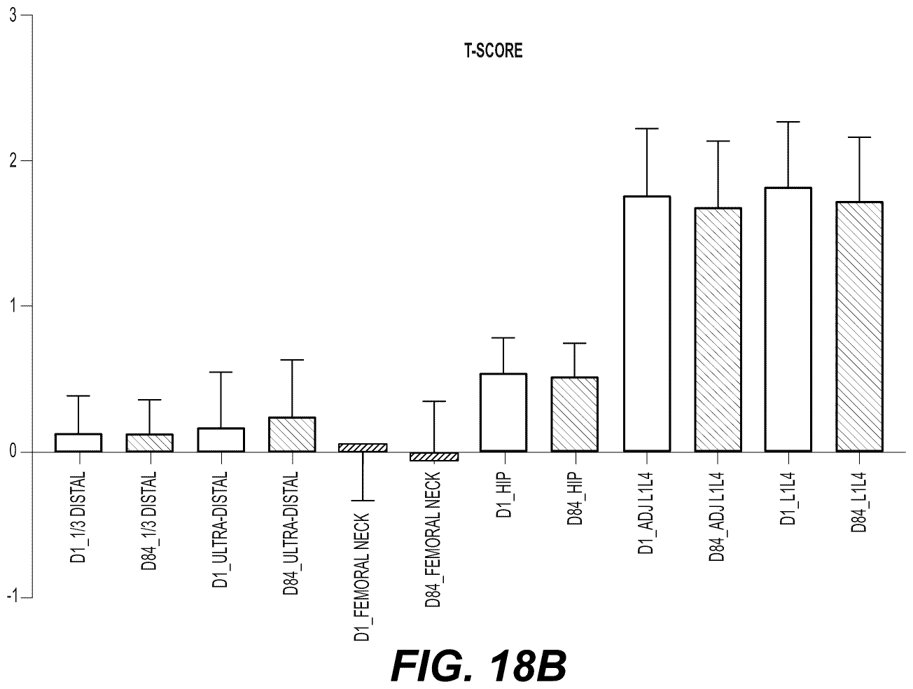

FIGS. 16A-16D show that treatment with AZP-3601 induced a gradual increase in both anabolic and catabolic bone markers to the mid-normal level by 4-8 weeks. The data presented therein also demonstrates that AZP-3601 never increased either mean bone marker above the upper limit of normal level, thereby supporting the working hypothesis that AZP-3601's mechanism of action targets urinary calcium reabsorption rather than bone resorption. This is a differentiator as up to 17% of patients with HP have osteopenia or osteoporosis; 53% are peri- or post-menopausal women. Bone mineral density (BMD) and trabecular bone score (TBS) remained stable in subjects of both cohorts (FIGS. 17A and 17B). These results demonstrate that administration of AZP-3601 does not compromise bone integrity. FIGS. 18A and 18B show the effect of AZP-3601 administration on Z-scores and T-scores at various bone sites. Consistent with a balanced increase in bone biomarkers, the data demonstrates that Z-score and T-score remain stable, including in patients with osteopenia. The T-score data further shows that 43% of the patients (6 patients out of 14 patients) were osteopenic at baseline at least at one anatomical site.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

### BIBLIOGRAPHY

1. Bilezikian J, ed. Primer on the Metabolic Bone Diseases and Disorders of Mineral Metabolism. 9th ed. Washington, DC: American Society for Bone and Mineral Research; 2019.
2. Gafni R I and Collins M T. Hypoparathyroidism. N Engl J Med. 2019; 380(18):1738-47.
3. Rubin M R, Dempster D W, et al. Dynamic and Structural Properties of the Skeleton in Hypoparathyroidism. 2008 December; 23(12):2018-2024.
4. Vokes T J. Quality of Life in Hypoparathyroidism. Endocrinol Metab Clin North Am. 2018; 47(4):855-64.
5. Mannstadt M, Clarke B L, et al. Efficacy and safety of recombinant human parathyroid hormone (1-84) in hypoparathyroidism (REPLACE): a double-blind, placebo-controlled, randomised, phase 3 study. Lancet Diabetes Endocrinol. 2013 December; 1(4):275-83.
6. Winer K K. Does PTH Replacement Therapy Improve Quality of Life in Patients With Chronic Hypoparathyroidism? J Clin Endocrinol Metab. 2018 Jul. 1; 103(7):2752-2755.
7. Bi R, Fan Y, Lauter K, et al. Diphtheria Toxin- and GFP-Based Mouse Models of Acquired Hypoparathyroidism and Treatment With a Long-Acting Parathyroid Hormone Analog. J Bone Miner Res. 2015; 31(5):975-84.
8. Shimizu M, Joyashiki E, Noda H, et al. Pharmacodynamic Actions of a Long-Acting PTH Analog (AZP-3601) in Thyroparathyroidectomized (TPTX) Rats and Normal Monkeys. J Bone Miner Res. 2016; 31(7):1405-12.
9. Aliya A Khan, Mishaela R Rubin, et al. Efficacy and Safety of Parathyroid Hormone Replacement With TransCon PTH in Hypoparathyroidism: 26-Week Results From the Phase 3. PaTHway Trial. J Bone Miner Res. 2023; 38(1):14-25.
10. Lars Holten-Andersen, Susanne Pihl, Caroline E Rasmussen, et al. Design and Preclinical Development of TransCon PTH, an Investigational Sustained-Release PTH Replacement Therapy for Hypoparathyroidism. J Bone Miner Res. 2019 November; 34(11):2075-2086.
11. Aliya A Khan, Mishaela R Rubin, Peter Schwarz, et al. Efficacy and Safety of Parathyroid Hormone Replacement With TransCon PTH in Hypoparathyroidism: 26-Week Results From the Phase 3 PaTHway Trial. J Bone Miner Res. 2023 January; 38(1):14-25.
12. Clarke B, Khan A A, Rubin M R, et al. Long-Term Efficacy and Safety of TransCon™PTH in Adults with Hypoparathyroidism: 52-Week Results From the Open-Label Extension of the Phase 3 PaTHway Trial. Paper presented at: the Endocrine Society annual meeting. Jun. 15-18, 2023.
13. Underbjerg et al. Postsurgical hypoparathyroidism—risk of fractures, psychiatric diseases, cancer, cataract, and infections. JBMR 29:2504, 2014.
14. Underbjerg et al., The Epidemiology of Nonsurgical Hypoparathyroidism in Denmark: A Nationwide Case Finding StudyJBMR 30:1738, 2015).
15. Chawla et al. Vertebral Fractures and Bone Mineral Density in Patients With Idiopathic Hypoparathyroidism on Long-Term Follow-Up. JCEM 102:251, 2017.
16. Mendonea et al. Increased vertebral morphometric fracture in patients with postsurgical hypoparathyroidism despite normal bone mineral density. Endocrine Disorders 13:1, 2013.
17. Tam et al. Parathyroid Hormone Stimulates the Bone Apposition Rate Independently of Its Resorptive Action: Differential Effects of Intermittent and Continuous Administration. Endocrinology 110:505, 1982.
18. Dobnig and Turner. The effects of programmed administration of human parathyroid hormone fragment (1-34) on bone histomorphometry and serum chemistry in rats. Endocrinology 138: 4607, 1997.
19. Horwitz et al. A 7-day continuous infusion of PTH or PTHrP suppresses bone formation and uncouples bone turnover. J Bone Miner Res 26:2287, 2011.
20. Holten-Andersen et al. Design and Preclinical Development of TransCon PTH, an Investigational Sustained-Release PTH Replacement Therapy for Hypoparathyroidism. JBMR 34:2075, 2019

What is claimed is:

1. A method for management and/or treatment of hypoparathyroidism (HP) in a subject, the method comprising administering a dose of a PTH compound to the subject, wherein the PTH compound has an amino acid sequence as set forth in SEQ ID NO: 10; and wherein administration of the PTH compound maintains or improves bone integrity.

2. The method of claim 1, wherein the dose of the PTH compound is a daily dose.

3. The method of claim 1, wherein the dose of the PTH compound is between 10 g/day and 120 μg/day.

4. The method of claim 1, wherein the dose of the PTH compound is between 10 g/day and 100 μg/day.

5. The method of claim 1, wherein the dose of the PTH compound is between 10 g/day and 80 μg/day.

6. The method of claim 1, further comprising titrating the subject off of a standard of care treatment for HP from the time a first dose of the PTH compound is administered.

7. The method of claim 6, wherein titrating the subject off of standard of care treatment for HP is performed within 12 weeks of the time the first dose of the PTH compound is administered.

8. The method of claim 6, wherein titrating the subject off of standard or care treatment for HP is performed within 10 weeks of the time the first dose of the PTH compound is administered.

9. The method of claim 6, wherein titrating the subject off of standard or care treatment for HP is performed within 6 weeks of the time the first dose of the PTH compound is administered.

10. The method of claim 6, wherein titrating the subject off of standard or care treatment for HP is performed within 4 weeks of the time the first dose of the PTH compound is administered.

11. The method of claim 6, wherein titrating the subject off of standard or care treatment for HP is performed within 2 weeks of the time the first dose of the PTH compound is administered.

12. The method of claim 6, wherein titrating the subject off of standard of care treatment for HP comprises decreasing administration of the standard of care treatment until the administration of the dose of the PTH compound results in a stable albumin-corrected serum calcium level in the subject.

13. The method of claim 12, wherein the stable albumin-corrected serum calcium level is between 8.3 mg/dL and 10.6 mg/dL mg/dl.

14. The method of claim 1, wherein the subject is a hypercalciuric subject.

15. The method of claim 14, wherein the method further normalizes urinary calcium levels in the hypercalciuric subject.

16. The method of claim 1, wherein the subject is a peri-menopausal or post-menopausal women.

17. The method of claim 1, wherein the subject suffers from osteopenia or osteoporosis.

18. The method of claim 1, wherein the administration is by subcutaneous injection.

19. The method of claim 18, wherein the subcutaneous administration is with a pen injector.

20. A pharmaceutical composition comprising a dose of a PTH compound having an amino acid sequence as set forth in SEQ ID NO: 10, wherein the dosage is between 10 μg/day and 120 μg/day.

21. An injectable pen comprising the pharmaceutical composition of claim 20.
